(19) United States
(12) Patent Application Publication
(10) Pub. No.: US 2004/0136957 A1
(43) Pub. Date: Jul. 15, 2004

(54) At least partial prevention and/or reduction of cellular damage in tissue that has suffered from or is suffering from hypoxia and/or ischaemia and/or inflammation

(75) Inventors: Wouter Bernard Veldhuis, Utrecht (NL); Petrus Hendrikus van der Meide, Nootdorp (NL); Klaas Nicolay, Houten (NL)

Correspondence Address: TRASK BRITT, P.O. BOX 2550, SALT LAKE CITY, UT, 84110, US

(73) Assignees: Universitair Medisch Centrum Utrecht, Utrecht (NL); Universiteit Utrecht Holding B. V., Utrecht (NL)

(21) Appl. No.: 10/678,957

(22) Filed: Oct. 3, 2003

Related U.S. Application Data
(63) Continuation of application No. PCT/NL01/00217, filed on Apr. 3, 2002.

(51) Int. Cl. A61K 38/21

(52) U.S. Cl. 424/85.7

(57) ABSTRACT

The present invention provides means and methods for at least a partial prevention of cellular damage in disease resulting from hypoxia/ischemia (H/I)-related inflammation, preferably due to occlusion of blood vessels. Administration of IFN type-I before, around or after the time of lifting of the ischemia results in improved survival of cells downstream of the occluded blood vessel, presumable because the H/I-related inflammation is attenuated. Treatment results in less damage, better recovery and/or improved repair of the affected tissue.

### CROSS-REFERENCE TO RELATED APPLICATION

This application is a continuation of PCT International Patent Application No. PCT/NL02/00217, filed on Apr. 3, 2002, designating the United States of America, and published, in English, as PCT International Publication No. WO 02/080953 A2 on Oct. 17, 2002, the contents of the entirety of which is incorporated by this reference.

### TECHNICAL FIELD

The invention relates generally to the field of medicine, more particularly to the treatment of diseases, especially diseases in which interruption of blood flow and/or activation/infiltration/proliferation of immune cells and/or inflammation has detrimental effects on tissue.

### BACKGROUND

Interruption of blood flow is detrimental to tissue and in fact is the cause of a variety of human disease. Restoration of blood flow, spontaneous and/or treatment-induced can theoretically salvage all or part of the affected tissue. The success of this reperfusion in preventing further tissue damage is amongst others dependent on the severity of blood flown decrease and the time to restoration of blood flow to levels compatible with cell survival.

Treatment directed to increase blood flow—and/or spontaneous restoration of blood flow—to therapeutically relevant levels and treatment directed to protect cells from the results of the blood flow interruption is often counteracted by a phenomenon that we refer to as hypoxia/ischemia (H/I) related inflammation. The H/I related inflammation adversely affects the survival of tissue after removal of the primary obstruction, or, when the obstruction has not (yet) been removed: the chance of survival of the tissue. This phenomenon is not only relevant in situations where blood supply is temporarily interfered with. A similar phenomenon is observed in situations where the demand for blood flow is (chronically) lower than the supply. Although the term hypoxia refers to situations of oxygen shortage, the phenomenon does not have to be related to the oxygen shortage. The term is here only used to indicate a situation in or after which the phenomenon is observed. The phenomenon may, for instance, also be induced by nutrient shortage. H/I related inflammation is a problem in a variety of human disease. The reasons for the detrimental effect of the phenomenon on the survival of tissue after removal of the primary obstruction are not known. The effects may be the primary or secondary result of inflammation. It may also be the result of resistance to the increased blood flow following removal of the obstruction or be due to the quality of the remaining perfusion. Inflammatory like responses are typically observed in these situations. Various associations and explanations for the inflammatory reaction have been proposed, however, as of yet this has not led to the development of improved therapies.

### BRIEF SUMMARY OF THE INVENTION

The present invention provides a method of treating H/I related inflammation in an individual comprising administering to the individual a therapeutically relevant dose of a type I interferon (IFN type-I) or a functional part, derivative and/or analogue thereof. Administration of the dose leads to reduced H/I related inflammation in the individual. The invention further provides a method for reducing cellular damage in tissue that has suffered from or is suffering from hypoxia and/or ischemia and/or inflammation in an individual comprising administering to the individual a therapeutically relevant dose of IFN type-I or a functional part, derivative and/or analogue thereof. Survival of tissue displaying H/I related inflammation in the untreated individual, is improved in the comparable tissue in the treated individual. Less damage, better recovery or improved repair of the affected tissue is observed. One explanation is that as a result of the administration of a type I interferon, the inflammatory response is attenuated. H/I related inflammation does not have to be displayed by the individual at the time of administration. Prophylactic application is also beneficial. I.e. when it is expected that H/I related inflammation can occur in an individual, the individual can be treated with a method of the invention and profit from the reduced damage, better recovery or improved repair of the affected tissue, as compared to the situation where no IFN type-I or functional part, derivative and/or analogue thereof, is given to the individual. As a non-limiting example this situation is illustrated for instance in surgery where one or more parts of the body may suffer from reduced blood flow due to isolation from the circulation.

With the phrase “treatment of H/I related inflammation” is meant that at least one effect of the phenomenon that we refer to as “H/I related inflammation” is at least in part reduced as a result of the treatment. Effects of the phenomenon are, for example, tissue damage, cell death or loss of function. Some or all of the results of the treatment may be due to the attenuation of the inflammatory response. Complete disappearance of, or protection against the inflammation as a result of treatment is possible.

H/I related inflammation can occur in many situations. A person that has not been able to breath oxygen for a limited amount of time, for instance, because the individual is in a situation where he/she has to be resuscitated, can be treated with a method of the invention and expect a beneficial effect on the overall outcome. Another likely embodiment of the invention is the treatment of traumatic brain injury (TBI), which can result from accidents like car crashes, falling, being hit on the head and the like. After traumatic brain injury blood flow is often reduced as a result of swelling of brain tissue or rupture of blood vessels or both. The present invention would improve the outcome by attenuating the H/I related inflammation following traumatic brain injury, and thus result in reduced damage, better recovery or improved repair of the tissue affected by the injury compared to the situation where no treatment is given.

In a preferred embodiment of the invention, the H/I related inflammation is the result of a shortage in blood supply. A situation where H/I related inflammation can occur is very frequently a local problem. Therefore typically, the H/I related inflammation affects a part of the body of the individual, preferably due to an obstruction of a blood vessel. An obstruction can be a complete obstruction of a vessel allowing no passage of blood. An obstruction can also be an incomplete obstruction of a vessel that disables the flow of blood to meet the demand of blood flow. An obstruction can be in-the form of a clot that clogs the vessel, or can be caused by the expansion of a lesion in the vessel wall or by a spasm of the vessel. However, an obstruction can also be the closing of a vessel by force exerted from the outside for instance due to clasping or swelling of surrounding tissue. An obstruction can also be caused by a cut effectively interfering with proper blood flow. A non-limiting example of the previous is a spontaneous rupture of a major supplying brain vessel, such as occurring in sub-arachnoid hemorrhage.

In one aspect, the invention therefore provides a method for the treatment of an H/I related inflammation in an individual, the individual comprising at least one blood vessel obstruction causing ischemia (the supply of blood lags behind the demand of blood, whether due to obstructions, increased demand, lowered level of oxygen and/or nutrients in the blood or any other way) in tissue that is situated downstream from the obstruction, the method comprising administering to the individual a therapeutic dose of IFN type-I or a functional part, derivative and/or analogue thereof. A treatment of the invention preferably results in improved survival of cells in the individual, preferably, the cells comprises neuronal cells. In another aspect, the invention provides a method for at least in part improving blood flow in post-ischaemic tissue, for example by attenuating the clogging of microvasculature by inflammatory cells adhering to the endothelium, comprising administering to an individual, having the tissue, IFN type-I or a functional part, derivative and/or analogue thereof. In yet another embodiment, the invention provides a method for at least in part preventing cell death in post-ischaemic tissue comprising administering to an individual, having the tissue, IFN type-I or a functional part, derivative and/or analogue thereof. Preferably, the cell death is in part prevented in neuronal tissue.

In a preferred aspect of the invention, the H/I related inflammation is restrictedly to the brain or spinal cord, the heart, a transplanted organ and/or a, limb. A method of the invention can be used to improve survival of transplanted organs or parts thereof. In organ transplantation, reperfusion injury causes tissue damage immediately following transplantation, probably due to the presence and/or invasion of inflammatory cells in the damaged area. This, process has been implicated in the development of acute and chronic rejection of transplants. NF-kB is a transcription factor that upregulates adhesion molecules, e.g. ICAM-1, VCAM-1, and E-selectin, following reperfusion. Systemic treatment with sulfasalazine, a potent inhibitor of NF-kB, was shown to decrease adhesion molecule expression, decrease reperfusion injury, and prolong allograft survival in rat cardiac transplants. IFN type-I reduces the expression of adhesion molecules, (e.g., ICAM-1 or VCAM-1), and can be administered to an individual according to a method of the invention to prolong allograft survival.

One explanation for at least part of the beneficial effect of IFN type-I in this embodiment is that IFN type-I inhibits the induced production of IL-8 via transcriptional inhibition of IL-8 gene expression (Oliveira et al., 1992; Oliveira et al., 1994). Ischemia-reperfusion injury in rat small intestine can be attenuated by administration of antibodies against IL-8 (Tsuruma et al., 1998).

In a preferred embodiment, the H/I related inflammation is restricted to a part of the brain and/or the heart. Particularly the brain is preferred because the administration of IFN type-I or a functional part, derivative and/or analogue thereof, has a dramatic effect on H/I related inflammation in this part of the body. Administration results in significantly less neuronal damage, increased cell survival and improved repair of the damaged area. The volume of the damaged area in the brain as a result of the administration is significantly reduced compared to untreated. One explanation for the observed effects of treatment is that inflammation in the affected area is attenuated significantly compared to untreated individuals.

In a preferred embodiment, IFN type-I or a functional part, derivative and/or analogue thereof, is administered to patients in whom the blood flow is insufficient in an area of the brain, preferably as the result of a stroke or trauma. In cases where an obstruction of a vessel is the reason for the insufficient blood flow it is preferred that the compound is administered as soon as possible, and preferably even before occurrence of the obstruction. Administration can prolong the time window during which the partial) removal of the obstruction is beneficial. In this case, administration can be continued until a more or less stable situation has been arrived at. In cases where hypoxic/ischaemic tissue is reperfused, or perfused to a larger extent, a stable situation typically is arrived at within one week after the increased supply of blood flow was initiated. Nevertheless, treatment for longer periods (up to several weeks), is beneficial by attenuating the more chronic phase of H/I related inflammation. More chronic effects could be mediated by downregulation of VCAM-1.

It is thought that in the brain, ischemia and trauma elicit an inflammatory response in which mediators (cytokines, chemokines and adhesion molecules) are released and immune cells infiltrate the injured brain. Upregulation of pro-inflammatory cytokines, chemokines and endothelial-leukocyte adhesion molecules starts soon after ischemia and trauma and continues during the evolution of tissue injury. For leukocytes to gain access to their target tissues, a variety of signaling proteins and adhesion molecules act in concert to permit chemotaxis, endothelial cell attachment and transmigration. For example a rapid overproduction of TNF-alpha leads to the stimulation of adhesion molecule expression with subsequent accumulation of leukocytes in the ischaemic focus. Focal adhesion of leukocytes to the endothelium is a step in inflammation and certain vascular disease processes: P- and E-selectin and ICAM-1, expressed by activated endothelium, and the leukointegrin CD11/CD18 expressed by activated leukocytes, have been shown to contribute to this adhesion. E-selectin and ICAM-1 are synthesized by stimulation with cytokines such as IL-1beta and TNF-alpha. Immunoreactivity to ICAM-1 and CD11/CD18 has been demonstrated after 60 min. transient focal ischemia in the rat, in both core and penumbra, increasing from 3 to 24 h after reperfusion. Leukocytic infiltration in this example was seen in the ischaemic areas from 12 to 24 h after reperfusion. As mentioned above, effects of IFN type-I observed in the present invention can be the result of a reduction of inflammation responses in the affected areas. A possible mechanism by which inflammation can be reduced is by reducing the role of leukocytes. According to this view, leukocyte involvement in the pathogenenesis of cerebral ischaemic damage is as follows: exacerbation of blood-brain barrier (BBB) or parenchymal injury via hydrolytic enzyme release, oxygen radical production and lipid peroxidation, and/or a reduction in cerebral blood flow by vessel plugging and/or release of vasoconstrictive mediators such as, for example, endothelin.

The present invention is suited for at least in part reducing the negative effects of traumatic brain injury. Administration of IFN type-I reduces the volume of the damaged area, decreases cell death and enhances the recovery and/or repair of damaged tissue compared to untreated individuals. One explanation for the beneficial effects in traumatic brain injury is that the blood-brain-barrier is fortified. Dysfunction of the blood-brain barrier is mediated by chemokine release (e.g., IL-8), upregulation of adhesion molecules (e.g., ICAM-1, VCAM-1) and intracerebral neutrophil accumulation. IFN type-I can directly downregulate IL-8 via transcriptional inhibition of IL-8 gene expression and downregulates adhesion molecules. In patients with severe TBI, it was found that elevated cerebrospinal fluid IL-8 and soluble(s) ICAM-1 correlated with BBB-dysfunction: IL-8 and sICAM-1 were upregulated for 19 days after severe TBI, whereas their stimulator TNF-alpha was upregulated for 9 days. The same holds true for critical limb ischemia.

The mechanisms behind the beneficial effects of IFN type-I administration may be different for different diseases, i.e., reduction in brain damage could at least in part be due to a different effect than beneficial effects in the treatment of listed diseases affecting other parts of the body.

IFN type-I has pleiotropic effects. Several possible ways in which IFN type-I might act are listed below.

IFN type-I stimulates IL-10 production: IL-10 is a classic anti-inflammatory cytokine. Administration of IL-10 alone is enough to reduce infarct size after permanent MCA-occlusion (Spera et al., 1998) and IL-10 plus mild hypothermia (but not hypothermia alone) provides long-lasting protection to CA1 hippocampus following transient global ischemia. (Dietrich et al., 1999). IFN type-I stimulates IL-10 production in an in vitro microglia-T-cell interaction model (Chabot and Yong, 2000). Liu et al., showed that monocytes in vitro and in vivo in MS patients produced more IL-10 following IFN type-I treatment (Liu et al., 2001). In a murine model of experimental autoimmune encephalomyelitis (EAE), an animal model sharing many features with MS, Tuohy et al. found that IFN type-I significantly skewed the response to the priming immunogen toward an increased production of IL-10 and a concurrent decreased production of IL-12 and that this was accompanied by an aborted development of epitope spreading (Tuohy et al., 2000). Others have shown that-in clinical MS, levels of IL-10-secreting monocytes were augmented during IFN type-I treatment (Ozenci et al., 1999) and that IL-10 levels were elevated during resolution of lesions in IFN type-I treated patients (Waubant et al., 2001b). IL-10 can suppress the production of a variety of pro-inflammatory molecules, including TNF-alpha, IL-1beta, interferon-gamma and IL-6 (Fischer et al., 2001; Plunkett et al., 2001; Szczepanik et al., 2001). It diminishes neutrophil chemotaxis via IL-8 suppression (Howard and O'Garra, 1992; Ehrlich et al., 1998), it interferes with macrophage activity through inhibition of macrophage inflammatory protein-1 (MIP-1) expression and deactivates monocytes (Geng et al., 1994).

IFN type-I counteracts the pro-inflammatory cytokines TNF-alpha and IL-1beta. Numerous studies have shown the deleterious properties of TNF-alpha and IL-1beta in ischaemic stroke (Barone and Feuerstein, 1999). IFN type-I may attain its neuroprotective potential by combatting these key inflammatory cytokines: it has been shown in vitro (Chabot et al., 1997; Chabot and Yong, 2000; Jungo et al., 2001; Ossege et al., 2001), in mice (Bosca et al., 2000) and in blood from MS patients (Gayo et al., 1999; Perini et al., 2000; Ossege et al., 2001) that IFN type-I counteracts TNF-alpha- and IL-1beta actions by reducing-their upregulation and by inducing the IL-1 receptor-antagonist, as well as inducing soluble TNF-alpha-receptor I and II (Perini et al., 2000).

IFN type-I attenuates expression of the chemokines IL-8, MCP-1, RANTES and MIP-1 alpha: IFN type-I has been shown to inhibit the induced production of IL-8 via transcriptional inhibition of IL-8 gene expression (Oliveira et al., 1992; Oliveira et al., 1994). IL-8 (also termed CINC in rats) is a key mediator of leukocyte recruitment after ischemia/reperfusion injury. The chemokine RANTES (regulated upon activation, normal T-cell expressed and secreted) triggers monocyte arrest on inflamed and atherosclerotic endothelium (von Hundelshausen et al., 2001), and Bona et al., showed in a neonatal hypoxia/ischemia model that mRNA induction for MIP-1 alpha and RANTES preceded the appearance of lymphocytes, microglia/macrophages, and natural killer cells in the infarct area (Bona et al., 1999). Zang et al., demonstrated that in vitro exposure of T cells to IFN type-I selectively inhibited mRNA expression for RANTES and MIP-1alpha and their receptor CCR5 and that T cell surface expression of CCR5 was significantly reduced in MS patients treated with IFN type-I, correlating with decreased T cell transmigration toward RANTES and MIP-1alpha (Zang et. al., 2001). Iarlori et al., showed that in vivo RANTES production is reduced in sera from MS patients treated with IFN type-I (Iarlori et al., 2000). Monocyte chemoattractant protein-1 (MCP-1) is downregulated by IFN type-I in experimental autoimmune neuritis (Zou et al., 1999) inhibiting: migration of inflammatory cells into the nervous tissue. It was demonstrated that. IFN type-I treatment abrogated RANTES-, MIP-1alpha- and MCP-1 induced migration of monocytes across a matrix of fibronectin, an extracellular matrix molecule and a major component of the endothelial basement membrane, through an effect on matrixmetalloproteinase-9 (MMP-9). (Stuve et al., 1997). IL-8 causes neutrophils to secrete MMP-9 (Masure et al., 1991). MIP-1alpha, MIP-1beta and RANTES enhance-T-cell-secretion of MMP-9. IFN type-I may not only reduce IL-8, MIP-1 alpha and RANTES, it has also been shown to upregulate the naturally occurring inhibitor of MMPs, TIMP-1 (Waubant et al., 2001a). Together these mechanisms prevent trafficking and infiltration of pro-inflammatory cells into the lesions site. Moreover. MMPs have been shown to be key players in thrombolysis-associated hemorrhagic transformation after focal ischemia (Sumii and Lo, 2002). Thus, the present invention, by reducing MMP-activity, is likely to reduce bleeding, either spontaneous or after thrombolysis, and can thus increase the safety and extend the time-window for thrombolytic therapy.

IFN type-I prevents the upregulation of several cell adhesion molecules: Not just via preventing the upregulation of pro-inflammatory cytokines but also by directly downregulating or inhibiting the increase in levels of ICAM-1 (on BMECs in vitro (Defazio et al., 2000; Trojano et al., 2000)), of MHC class-II molecules (which might also act as lymphocyte adhesion receptors) (Huynh et al., 1995; Miller et al., 1996) and of Beta1-integrin (or VLA-4) (Soilu-Hanninen et al., 1995; Muraro et al., 2000). Furthermore, it has been shown that increasing soluble VCAM-1 (sVCAM) release during active inflammation could be a mechanism by which IFN type-I treatment exerts protective effects in multiple sclerosis patients (Kallmann et al., 2000; Calabresi et al., 2001) since sVCAM can bind and downregulate Beta1 integrin thus interfering with lymphocyte-adhesion to mVCAM (Calabresi et al., 1997).

IFN type-I decreases iNOS activity: Induction of iNOS is a well known aggravating factor mediating delayed ischaemic brain damage and its selective inhibition has been shown to be neuroprotective. Hua et al. found that in primary human astroglial cell cultures IFN type-I was a selective and potent inhibitor of IL-1beta- or interferon-gamma-induced iNOS expression (Hua et al., 1998). The same was found in a human astrocytoma cell line (Guthikonda et al., 1998) and murine macrophages (Deguchi et al., 1995; Lopez-Collazo et. al., 1998).

IFN type-I upregulates HIF-1alpha and TGF-beta: Der et al., illustrated the pleiotropic nature of IFN type-I using oligonucleotide-arrays in a human HT1080 cell line, identifying over 300 genes that are upregulated at least twofold after IFN type-1-stimulation (Der et al., 1998). Amongst these: hypoxia inducible factor-1alpha, (HIF-1alpha), which, under hypoxic conditions, activates the transcription of genes encoding erythropoietin, glucose transporters, glycolytic enzymes, vascular endothelial growth factor (VEGF), and other genes whose protein products increase O₂ delivery or facilitate metabolic adaptation to hypoxia. HIF-1alpha signaling might, however, also play a role in p53-mediated apoptotic cell death (Halterman et al., 1999) which seems to indicate that HIF-1alpha only induces transcription of protective genes in more penumbral regions where hypoxic stress is not too severe. IFN type-1-alpha induced the mRNA expression of TGF-beta and TGF-beta-R-II in vivo (Ossege et al., 2001) and acutely increased blood levels of TGF-beta (Nicoletti et al., 1998). TGF-beta displays multiple anti-inflammatory activities; pretreatment reduced infarct size and increased cerebral blood flow in rabbit stroke model (Gross et al., 1993) and intraventricular injection 2 hours after hypoxic-ischaemic injury reduced cortical infarction in the rat (McNeill et al., 1994).

The reasonings given above are given only to provide the reader with a possible mechanism. The striking effects of IFN type-I or a functional part, derivative and/or analogue thereof on reducing damage and improving recovery and/or repair of tissue affected by H/I related inflammation, as demonstrated in the present invention, can also be caused through an entirely different mechanism.

The invention further provides a use of IFN type-I or a functional part, derivative and/or analogue thereof for the preparation of a medicament for the treatment of H/I related inflammation. Preferably, the inflammation is correlated with hypoxia and/or ischemia. More preferably, wherein the impairment is in the brain.

### BRIEF DESCRIPTION OF THE SEVERAL VIEWS OF THE DRAWINGS

FIG. 1. ADC maps of a slice through the center of the lesion acquired on day 1, 7 and 21. Top row: Animal treated, with s.c. injections of saline. Bottow row: Animal treated with s.c. injections of 500.000 IU rrIFN-β, starting at 3 h after reperfusion.

FIG. 2. T₂ maps of a slice through the center of the lesion acquired on day 1, 7 and 21. Top row: Animal treated with s.c. injections of saline. Bottow row: Animal treated with s.c. injections of 500.000 IU rrIFN-β, starting at 3 h after reperfusion.

FIG. 3. Lesion volume of the control group and all treatment groups, on days 1, 7 and 21 as calculated from ADC maps.

FIG. 4. Lesion volume of the control group versus all treatment groups together, on days 1, 7 and 21 as calculated from ADC maps.

FIG. 5. Mean lesion ADC value of the control group and all treatment groups, on day 1 as calculated from ADC maps. All values were decreased compared to normal tissue (0.00073 mm²/s); control animals showed a stronger decrease in lesion ADC value.

FIG. 6. Mean lesion ADC value of the control group and all treatment groups, on days 7 and 21 as calculated from ADC maps. All values were increased compared to normal tissue (0.00073 mm²/s); control animals showed a stronger increase in lesion ADC value.

FIG. 7. Lesion volume of the control group and all treatment groups, on days 1, 7 and 21 as calculated from T₂ maps.

FIG. 8. Lesion volume of the control group versus all treatment groups together, on days 1, 7 and 21 as calculated from T₂ maps.

FIG: 9. Mean lesion T₂ value of the control group and all treatment groups, on days 1, 7 and 21 as calculated from T₂ maps. All values were increased compared to normal tissue (0.055 s); control animals showed a stronger increase in lesion T₂′ value.,

FIG. 10. Mean lesion T₂ value of the control group versus all treatment groups together, on days 1, 7 and 21 as calculated from T₂ maps. All values were increased compared to normal tissue (0.055 s); control animals showed a stronger increase in lesion T₂ value.

FIG. 11. ADC and T₂ maps of a slice through the center of the lesion acquired on day 1, 7 and 21. Top rows: ADC maps of an animal treated with s.c. injections of saline and of animal treated with s.c. injections of 500.000 IU rrIFN-beta, starting at 6 h after stroke onset. Bottom rows: T₂ maps of the same two animals.

FIG. 12. Lesion volume of the control group and all treatment groups, on days 1, 7 and 21, as calculated from ADC maps. Since there was no significant difference between any of the IFN-treatment groups over time, the mean effect of-IFN treatment is shown as IFN-mean. Asterisks denote significance of difference from control.

FIG. 13. Mean lesion ADC value of the control group and all treatment groups, on day 1 as calculated from ADC maps. Irrespective of the size of the lesion, all mean lesion ADC values were decreased compared to normal tissue (0.00073 mm²/s); control animals showed a stronger decrease in lesion ADC value.

FIG. 14. Mean lesion, ADC value of the control group and all treatment groups, on days 7 and 21 as calculated from ADC maps. Irrespective of the size of the lesion, all mean lesion ADC values were increased compared to normal tissue (0.00073 mm²/s); control animals showed a stronger increase in lesion ADC value.

FIG. 15. Lesion volume of the control group and all treatment groups, on days 1, 7 and 21 as calculated from T₂ maps. Since there was no significant difference between any of the IFN-treatment groups over time, the mean effect of IFN treatment is shown as IFN-mean. Asterisks denote significance of difference from control. Ampersand denotes significance of difference between prophylactic and post-hoc treatment.

FIG. 16. Mean lesion T₂ value of the control group and all treatment groups, on days 1, 7 and 21 as calculated from T₂ maps. Irrespective of the size of the lesion, all mean lesion T₂ values were increased compared to normal tissue (0.054 s); control animals showed a stronger increase in lesion T₂ value.

FIG. 17. Effect of IFN on EBB integrity assessed 24 h after stroke. The integrity of the BBB was better preserved in the cortex of IFN-treated animals, than in control animals as evidenced by a 49% decrease in signal GdDTPA-induced enhancement. Asterisk, P<0.05 versus ischaemic control; Bars represent means±SD.

FIG. 18. Effect of IFN on ICAM-1 expression assessed 24 h after stroke. Shown are sections through the ipsi- and contralateral hemispheres of IFN-treated animals (top row) and control animals (bottom row). A low level of ICAM-1 expression was seen in the contralateral hemisphere of both control and IFN-treated animals. ICAM-1 expression was upregulated after, the ischaemic episode in the ipsilateral hemisphere of control animals and the upregulation was reduced by IFN treatment.

FIG. 19. Effect of IFN on VCAM-1 expression assessed 24 h after stroke. Shown are sections through the ipsi- and contralateral hemispheres of IFN-treated, animals (top row) and control animals (bottom row). A low level of VCAM-1 expression was seen in the contralateral hemisphere of both control and IFN-treated animals. VCAM-1 expression was upregulated after the ischaemic episode in the ipsilateral hemisphere of control animals and the upregulation was reduced by IFN treatment.

FIG. 20. Effect of IFN on MHC-II expression assessed 24 h after stroke. Shown are sections through the ipsi- and contralateral hemispheres of IFN-treated animals (top row) and control animals (bottom row). No MHC-II expression was seen in the contralateral-hemisphere of both control and IFN-treated animals. MHC-II expression was upregulated after the ischaemic episode in the ipsilateral hemisphere of control animals and the upregulation was reduced by IFN treatment.

FIG. 21. Effect of IFN on neutrophil infiltration assessed 24 h, after stroke. Shown are sections through the ipsi- and contralateral hemispheres of IFN-treated, animals (top row) and control animals (bottom row). No neutrophil infiltration was seen in the contralateral hemisphere of both control and IFN-treated animals. Neutrophils infiltrated the ipsilateral hemisphere of control animals and the infiltration was reduced by IFN treatment.

### DETAILED DESCRIPTION OF THE INVENTION

In yet another aspect, the invention provides a use of IFN type-I or a functional part, derivative and/or analoguer thereof for the preparation of a medicament for the treatment, of impairment of blood flow recovery. Preferably, the impairment is correlated with hypoxia and/or ischemia. Preferably, the impairment is in a capillary vessel. Preferably, the impairment is in the brain.

The invention further provides a use of IFN type-I or a functional part, derivative and/or analogue thereof for the preparation of a medicament for at least in part preventing cell death in post-ischaemic tissue. Preferably, wherein the impairment is, in the brain.

The present invention also provides means and methods for at least in part preventing ischemia-related damage to tissue. Reduction in tissue damage is, possibly contributed to by, the result of reduced micro-vascular clogging, which occurs in situ in microvessels downstream from the site of occlusion. Reduced micro-vascular clogging prevents deterioration of blood flow in the microvasculature downstream of the ischemia upon the (partial) removal of the primary occlusion. In one aspect the invention therefore provides a method for at least in part improving blood flow in tissue that has suffered or is suffering from ischemia, comprising administering to an individual, having the tissue, IFN type-I or a functional part, derivative and/or analogue thereof. IFN type-I or a functional part, derivative and/or analogue thereof may be administered before the cause for the ischemia is removed. Preferably, the IFN type-I or a functional part, derivative and/or analogue thereof is given as soon as possible, preferably together or immediately after treatment given to (partially) remove the primary occlusion is also possible.

The present invention is particularly suited for reducing damage and improving recovery, and/or repair of the affected tissue in the brain. The volume of the damaged area is decreased compared to untreated cases. In addition the amount of in particular, neural cell death is decreased. IFN type-I administration is suited for the treatment of effects, caused by any type of violence to the head, including but not limited to traumatic brain injury frost damage, stroke, and intracranial bleedings (e.g.: subarachnoid hemorrhage, thrombolytica-induced, etc.). IFN type-I or the functional part, derivative and/or analogue thereof is preferably administered as soon as possible after the cause of the damage has taken place. In this way, medication or other methods to remove the obstruction is given a larger time window for effect. Thus improving the chances of recovery for a patient. Administration may also be in anticipation of the cause or as prophylactic. In some diseases caused by occlusion of blood vessels, the current perception is that there is no urgency in giving treatment since there is no effective way to prevent the tissue damage. An example of such a disease is stroke. Very often, the fact that a patient had a stroke is not considered a reason for immediate treatment of the patient.

It is generally thought that damage inflicted by the stroke is already irreversible by the time a patient is hospitalized and available for treatment. With the present invention it is apparent that there is a good reason for rapid treatment of patients. With the means and methods of the invention it is possible to at least in part reduce the extent of tissue damage. The invention therefore further provides a method for at least in part preventing cell death in post-ischaemic tissue comprising administering to an individual, having the tissue, IFN type-I or a functional part, derivative and/or analogue thereof.

IFN type-I is currently used in the chronic treatment of multiple sclerosis (MS) patients and has been reported to cause systemic side-effects in this patient group. The systemic reaction is usually described as flu-like. Fever, chills, muscle aches, sometimes headaches, often commence a few hours after the drug is injected and last for some hours. For non-chronic cases, administration of IFN type-I can be discontinued after a relatively short period, often no longer than 1 week after the onset of the disease. In situations where prolonged administration is desired the side effects of the treatment can be tolerated.

Type I interferons encompass interferon alpha, interferon beta, interferon omega, and so-called consensus type interferon or hybrid types interferon (see, for review, Viscomi GC (1997) Structure-activity of type I interferons. Biotherapy 10:59.86.) In a preferred embodiment, the IFN type-I comprises interferon β or a functional part, derivative and/or analogue thereof.

A “functional part” of IFN type-I is a part of IFN type-I comprising the same post-ischaemic damage reduction activity in kind as IFN type-I itself. The amount of activity of such a part may differ from the activity of the complete protein.

A person skilled in the art is capable of generating a suitable “derivative” of IFN type-I Derivatives can be, for instance, be obtained, by conservative amino acid substitution. Indeed currently prescribed, human interferons differ slightly in: amino acid sequence from natural human interferons. A suitable part of IFN type-I is for instance a part with an altered glycosylation pattern or a part that is non-glycosylated. Glycosylation can be prevented by removing or altering a glycosylation site of the molecule. If the generation of such a (partially) deglycosylated IFN type-I requires alteration of the amino acid composition than such a deglycosylated IFN type-I is derivative of a functional part of IFN type-I. A functional part derivative and/or analogue of IFN type-I comprises the same activity in kind not necessarily in amount.

In general, IFN type-I may modulate the profile of cytokine production toward that of the anti-inflammatory phenotype (for instance by upregulation of IL-10), and this appears to occur in the systemic circulation and within the CNS.

For suitable doses, ranges and formulations of IFN type-I or a functional part, derivative and/or analogue thereof, one may turn to literature regarding the use of the compound in humans in other disease areas such as multiple sclerosis. Additionally, dose finding studies can give suitable therapeutic doses for the present invention. By way of example a dose of 8M IU Betaseron, s.c., every other day; a currently accepted dosage regime for relapsing remitting MS. A 1.6 M IU dosage every other day was well tolerated by the patient (Arnason, 1999). Furthermore, up to 176 million IU (5.5 mg of protein) has been injected i.v. in patients, three times a week, without severe side-effects. This is over three times higher than the dose per kg bodyweight demonstrated to be effective in the present invention after s.c. injection. Moreover, since also the bio-availability of about 50% of s.c. injected IFN type-I is much lower than that of directly i.v. injected IFN type-I, there is a broad treatment window within which IFN type-I, administration can be varied to reach optimal individual effect.

The administration of IFN type-I or a functional part, derivative and/or analogue thereof, can be combined with one or more other compounds. Preferably, the compound blocks the upregulation of adhesion molecules on endothelium or on the cells they would bind. However, antibody against these receptors, shielding it and preventing binding or a cytokine-antagonist that inhibits the activity of cytokines regulating adhesion molecule expression or cytokines that mediate chemotaxis is also suitable. IFN type-I may also be combined with thrombolytic therapy. Attenuation of H/I related inflammation can add to the effectiveness of thrombolytic therapy and extend the time window for such treatment. For example, Zhang, et al., found that co-treatment of rtPA with either anti-ICAM-1 antibody or with antileukocyte adhesion antibody (anti-CD18) resulted in a significant reduction in lesion volume compared with rats treated with rtPA only (Zhang et al., 1999a; Zhang et al., 1999b). The findings of Bowes et al., with antibodies against the same adhesion molecules in a rabbit cerebral, embolism stroke model suggested that adjuvant anti-leukocyte adhesion-therapy increases the postischaemic duration at which thrombolytic therapy remains effective (Bowes et al., 1995). Therefore, in a preferred embodiment IFN type-I or a functional part, derivative and/or analogue thereof is combined with treatment given to remove an obstruction from a vessel. Preferably, the treatment comprises administration of a thrombolyticum, such as for example tPA or similar compounds, or ultra-sound-mediated clot-removal.

In another preferred embodiment, IFN type-I is given together with neuroprotective drugs, such as erythropoietin or dihydroascorbic acid. Combinations may exert a synergistic effect on tissue damage reduction, cell survival and repair of tissue. In yet another preferred embodiment IFN type-I is given together with drugs that can ameliorate the side-effects of IFN type-1-treatment.

All type-I interferons (interferon-alpha, interferon-beta, interferon-omega, hybrids and consensus version thereof) share a common receptor through which at least some of their effect are mediated (i.e. composed of the a/0 interferon receptor (IFNAR)-1 and IFNAR-2 chains (Uze et al., 1990, Cell 60,225-234; Novick et al., 1994, Cell 77, 391-400; Domanski et al. 1995, J. Biol. Chem. 270, 21606-21611). The type-1-interferon-receptor can be targeted to develop compounds with similar activity as type I interferons. For the present invention such compounds are considered analogues of IFN type-I. In the present invention interferon-beta was used to demonstrate the effectiveness of activating the IFN type-1-R1, but activating the receptor using any other compound (for example, interferon-alpha) is also-effective.

Effectiveness of IFN type-I has been demonstrated using recombinant rat-IFN type-I in rats. The same recombinant rat-IFN type-I is also effective against experimental MS in rats, murine-IFN type-I is effective against MS in mice, both human- and primate-IFN type-I is effective against experimental MS in primates and recombinant human-IFN type-I has shown efficacy against MS in humans. Also, the occasional occurrence of a necrotic patch at the site of IFN type-I injection in rats, is a side-effect also seen in humans These lines of evidence indicate a conserved mode of action of IFN type-I in these different species.

In one aspect the invention provides a use of IFN type-I or a functional part, derivative and/or analogue thereof for the preparation of a medicament for the treatment of H/I related inflammation. The invention also provides a use of IFN type-I or a functional part, derivative and/or analogue thereof for the preparation of a medicament, the medicament capable of at least in part preventing cell death in post-ischemic tissue. Preferably, the impairment is in microvessels. More preferably, wherein the impairment is in the brain.

The invention is further explained with the aid of the following illustrative examples.

### EXAMPLES

### Example 1

Materials and Methods

Animal Model

Male Fischer rats (F344/Ico, Iffa-Credo Broekman, Someren, The Netherlands) aged 8-12 weeks were used in all experiments. Animals had free access to standard laboratory chow and water. Anesthesia was induced by i.p. injection of a mixture of 0.5 ml/kg fentanyl citrate (0.315 mg/ml) and fluanisone (10.0 mg/ml), and 0.5 ml/kg midazolam (5.0 mg/ml), followed by s.c. injection of 0.1 ml/kg (0.5 mg/ml) atropine sulfate and i.p. injection of 0.5 ml/kg gentamicinsulfate (10 mg/ml). The animals were endotracheally intubated and mechanically ventilated with O₂/N₂O (30/70 v/v). Body temperature was maintained at 37.0±0.5° C. by means of a feedback-controlled heating pad. During surgery and NMR-measurements anesthesia was continued by adding 1-1.5% halothane to the O₂/N₂O mixture. After surgery animals received 1 ml/kg buprenorfine s.c. (0.3 mg/ml).

Transient focal ischemia was induced by unilateral tandem occlusion of the right common carotid artery (CCA) and middle cerebral artery (MCA) via a modification of the procedure as described by Brint et al. (1988). Briefly, a 2 mm Ø hole was drilled just rostral to the oval foramen, exposing the right middle cerebral artery (MCA). After opening the dura and arachnoidea the MCA was transiently occluded using a vascular microclip (Codman). Reperfusion was reinstated after 60 minutes under visual inspection by removing the clip.

Animals were treated with s.c. injections of 500.000 IU rrIFN-β in 1 ml phosphate buffered saline (PBS) once daily until 7 days after reperfusion (n=9). Control animals (n=5) received s.c injections with saline, starting 2 days prior to surgery up to 7 days after: reperfusion. Treated animals were divided into three groups (n=3×3): treatment for group 1 started 2 days prior to surgery, treatment for group 2 started upon reperfusion and treatment for group 3 started 4 hours after stroke onset.

NMR Experiments

NMR-experiments were performed on a 4.7T Varian horizontal bore, spectrometer equipped with a gradient insert able to achieve gradients up to 220 mT/m in 300 μs. Rf-excitation and signal detection were accomplished by means of a Helmholtz volume coil (9 cm Ø) and an inductively coupled surface coil (2 cm Ø), respectively. Animals were positioned in an animal cradle and immobilized with ear bars. During the experiments exhaled CO₂ and rectal temperature were continuously monitored.

A single-scan diffusion-trace MRI-sequence (4 b's:100-1780 s/mm², repetition time (TR)=2 s, echo time (TE)=100 ms, number of transients (NT)=2); was used to generate quantified images of tissue water trace apparent diffusion coefficient (ADC). T₂-weighted images were acquired using a multi-echo sequence (8 TEs: 17.5 ms+7×17.5 ms, TR=2 s, NT=2).

Diffusion-weighted and T₂-weighted-data sets (collecting 8 1.7 mm thick slices, 3.2×3.2 cm² FOV, 128×64 matrix, zero-filled to 256×256) were acquired at 1, 7 and 21 days after reperfusion.

Data Analysis

ADC and T₂ maps were generated by mono-exponential fitting using IDL (Research Systems, Boulder, USA). Parametric images were analysed in anatomic regions of interest using in-house software. Calculations of volumes of affected tissue were based on ipsilateral ADC or T₂ differing more than 20% (corresponding to >2×SD) from the mean value in the contralateral hemisphere. Statistical analysis was carried out using SPSS 9.0. Reported p-values correspond to two-tailed Student's t-tests-or one-way ANOVA where-appropriate.

Results

General Condition

After surgery, body weight of all animals showed an initial decrease in the first postoperative days, whereafter it increased. A

In two treated animals a necrotic patch, about 7.5 mm in diameter, developed in the skin around the site of ifn-injection. The lesion was not painful to the touch and did, not hinder the animals in feeding or other behavior. For the remaining-days injection was continued at a different site; where, in these two animals, necrosis did not occur.

Magnetic Resonance Imaging

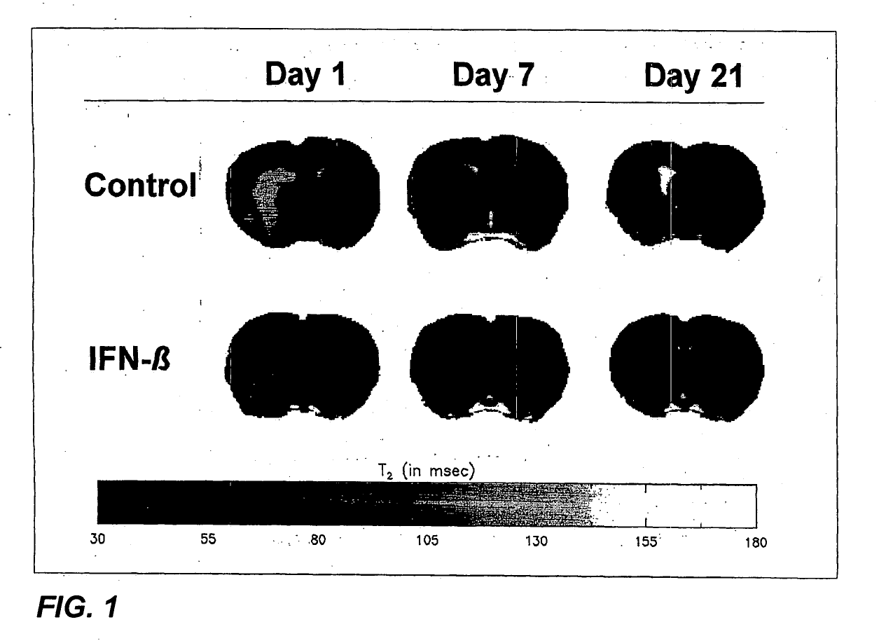

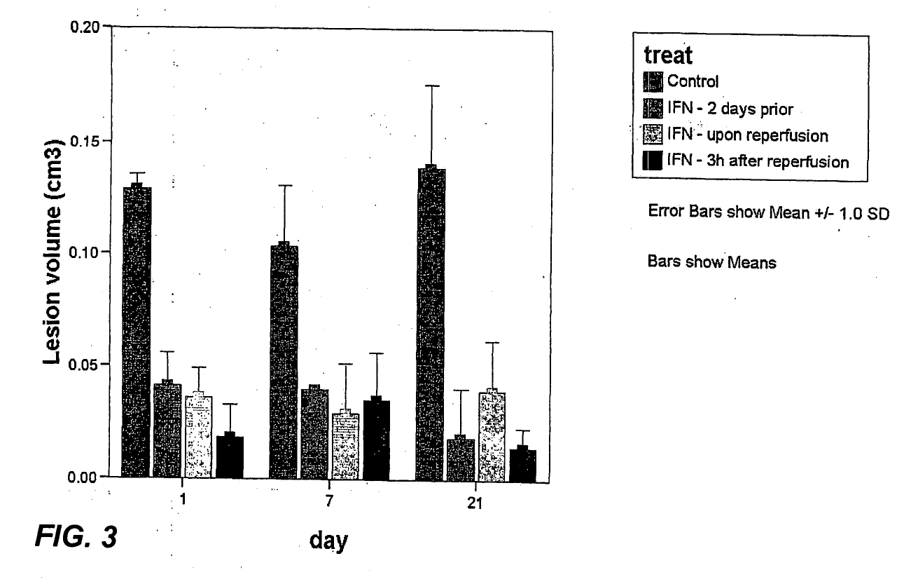

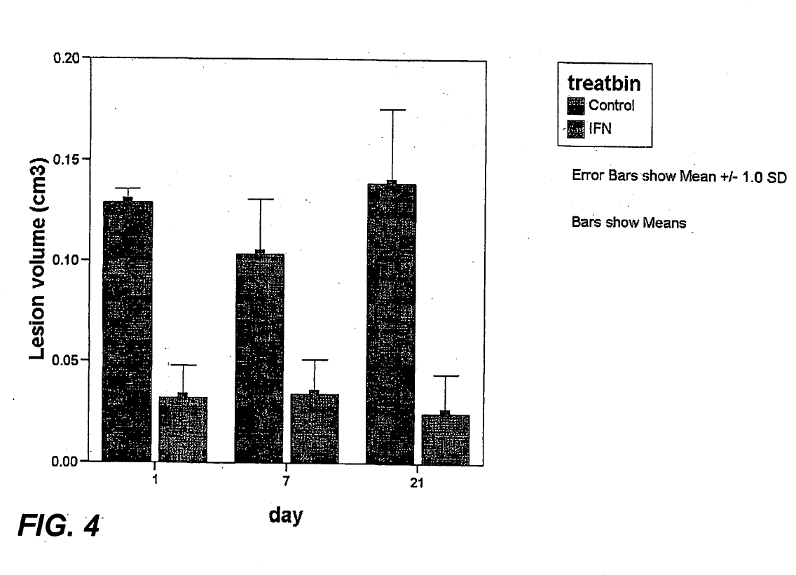

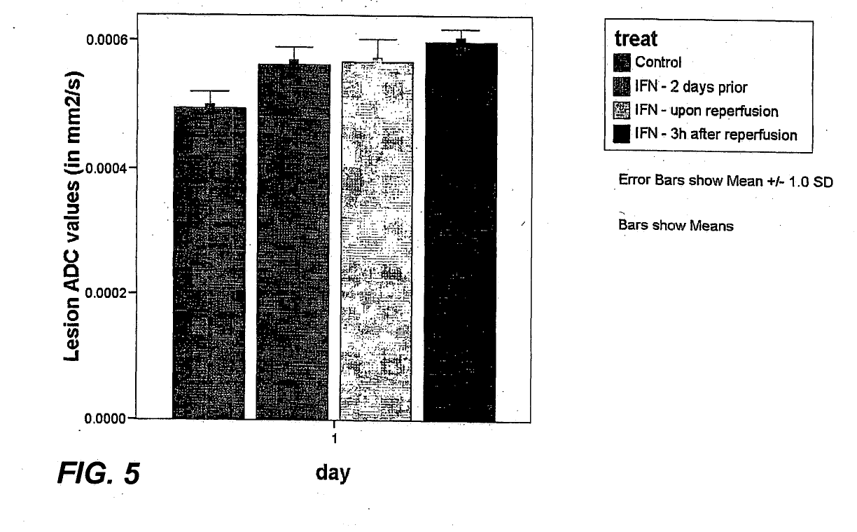

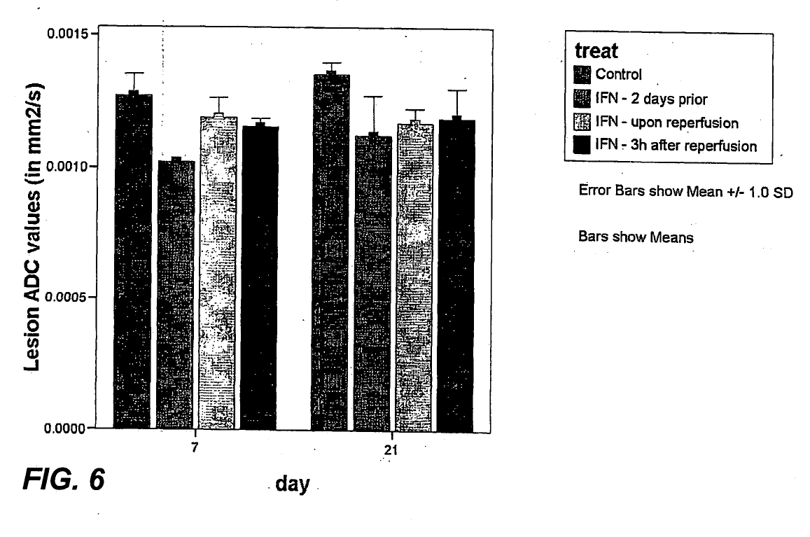

-ADC images acquired at 1 day after reperfusion showed cytotoxic cell swelling, as evidenced by decreased ADC-values. In control animals both the cortex and a large part of the caudateputamen of the ipsilateral hemisphere were affected, while in interferon-treated animals a smaller part of the caudateputamen and no part or only a small rim of cortext showed cytotoxic cell swelling (see, FIG. 1). At this timepoint the volume of the lesion on ADC maps (ALV) was 76% smaller (p<0.0005) in treated animals compared to controls (see FIGS. 3 and 4). Also, in interferon-treated animals the severity of tissue damage, as determined from the severity of ADC-reduction, was 15% less (p<0.002) than in control animals (see FIG. 5). There: was no significant difference between the three treatment, groups in terms of the T₂ data.

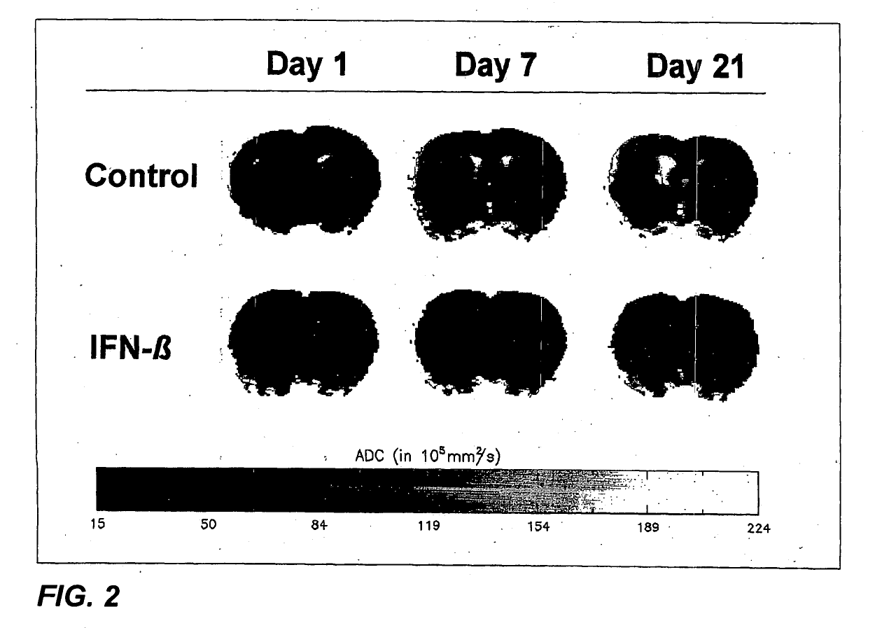

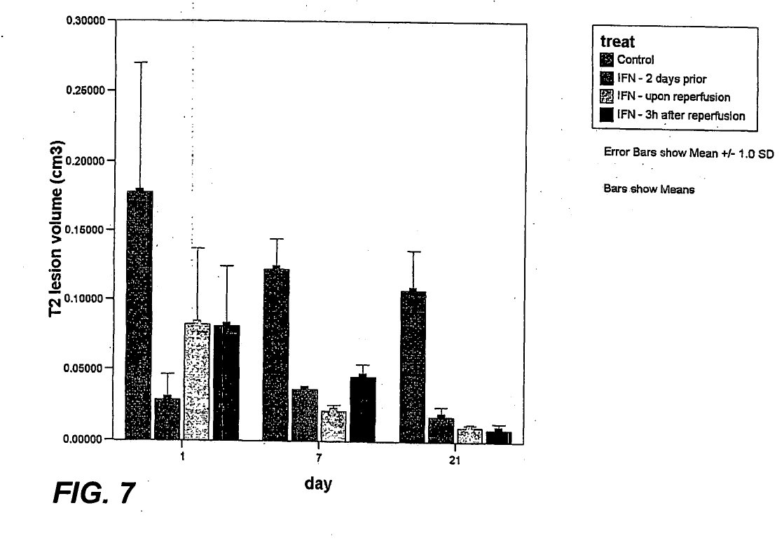

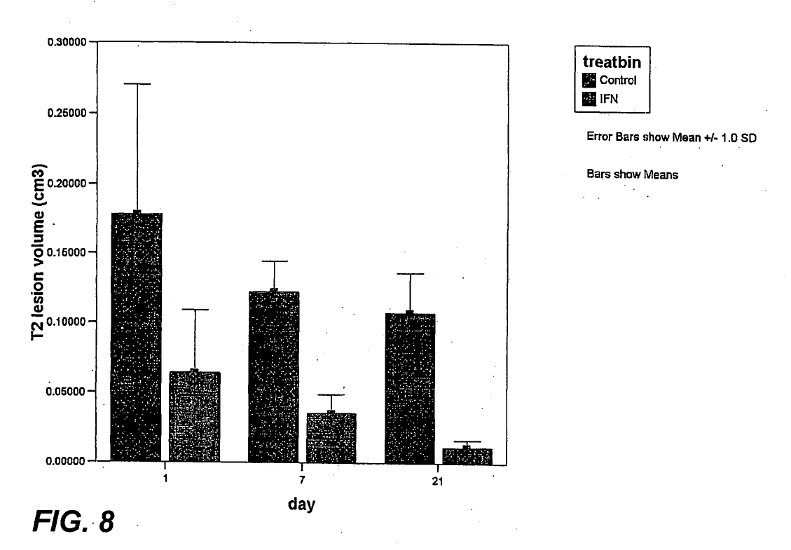

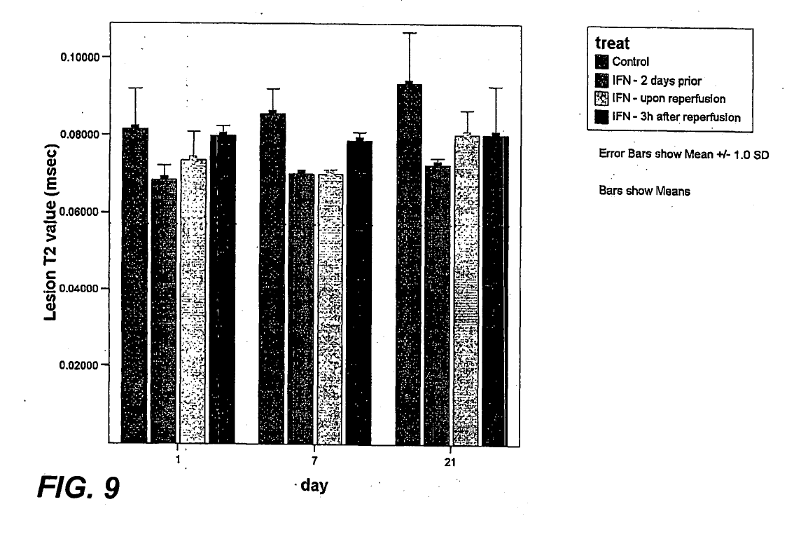

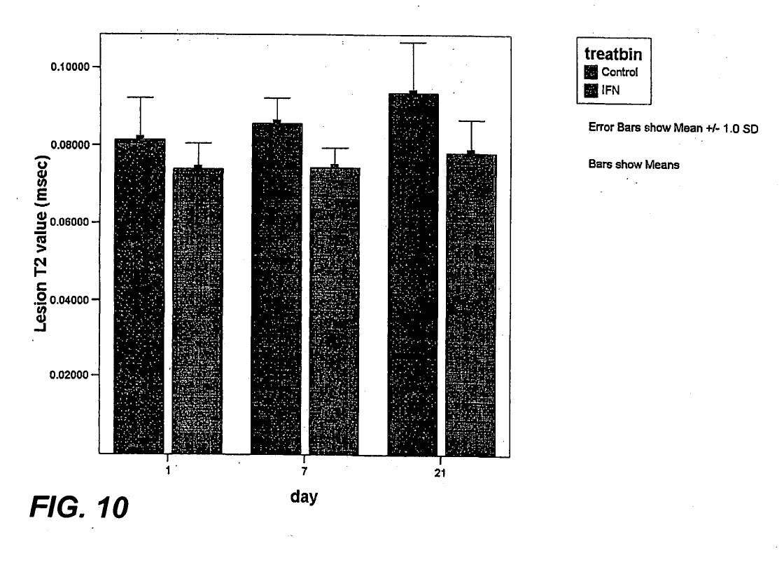

T₂ maps acquired at day 1 showed the development of vasogenic edema in the affected tissue, the volume of which was 64% smaller (p<0.021) in treated animals compared to controls (see, FIGS. 2, 7, and 8). This difference is also demonstrated by the midline-shift caused by swelling of the brain tissue, which is absent or much less, pronounced in treated animals compared to controls (see, FIG. 2). There was no significant difference between the three treatment groups.

ADC data acquired during the endpoint measurement on day 21 showed ADC values had increased to supranormal values, indicative of vasogenic edema and tissue loss (see FIGS. 1, 3 and 4). At this timepoint the infarct as present on ADC maps was reduced by 82% p<0.0005) in treated animals compared to controls.

T₂ map data acquired on day 21 confirmed the presence of vasogenic edema and tissue loss (see, FIGS. 2, 7 and 8). Infarct size as determined from T₂ maps was 89% smaller (p<0.0005) in interferon treated animals compared to controls.

To summarize, treatment with interferon-beta affords important neuroprotection to ischaemic brain. Already at 1 day after reperfusion, the amount of tissue at risk to undergo permanent infarction identified by the ADC data, is significantly smaller in interferon-treated animals than in control animals. Permanent damage as assessed by T₂ MRI shows the protection afforded at day 1 extends until at least 3 weeks after the insult, at which timepoint the volume of infarcted tissue is 89% smaller in treated animals compared to controls.

There was no significant difference between starting treatment 0.2 days prior to the insult, starting upon reperfusion or starting 4 hours after stroke onset, the latest timepoint tested, indicating a treatment window of 4 hours after onset of stroke and possibly-longer.

### Example 2

In this Example, the number of experimental animals, per group has been expanded compared to Example 1. Example 2 further contains additional data. Statistical analysis in this example was performed on all animals tested in Example 1 and Example 2.

Materials and Methods

Animal Model

Male Fischer rats (F344/Ico, Iffa-Credo Broekman, Someren, N L) aged 8-12 weeks were used in all experiments. Animals had free access to standard laboratory chow and water. Anesthesia was induced by i.p. injection of a mixture of 0.5 ml/kg fentanyl citrate. (0.315 mg/ml) and fluanisone (10.0 mg/ml), and 0.5 ml/kg midazolam (5.0 mg/ml), followed by sic. injection of 0.1 ml/kg (0.5 mg/ml) atropine sulfate and. i.p. injection of, 0.5 ml/kg gentamicinsulfate (10 mg/ml). The animals were endotracheally intubated and mechanically ventilated with O₂1N₂O (30/70 v/v). Body temperature was maintained at 37.0±0.5° C. by means of a feedback-controlled heating pad. During surgery and NMR-measurements anesthesia was continued by adding 1-1.5% halothane to the O₂/N₂O mixture. After surgery animals received 1 ml/kg buprenorfine s.c. (0.3 mg/ml).

Transient focal ischemia was induced by unilateral tandem occlusion of the right common carotid artery (CCA) and middle cerebral artery (MCA) via a modification of the, procedure as described by Brint et al. Briefly, a 2 mm Ø hole was drilled just rostral to the oval foramen, exposing the right middle cerebral artery (MCA). After opening the dura and arachnoidea the MCA was transiently occluded using a vascular microclip (Codman). Perfusion, was reinstated after 60 minutes under visual inspection by removing the clip.

Animals were treated with s.c. injections of 500,000 IU recombinant rat interferon-(˜8 μg of protein) in 1 ml phosphate buffered saline (PBS) once daily until 7 days after reperfusion (n=17). Control animals (n=12) received s.c injections with saline, starting; 2 days prior to surgery up to 7 days after reperfusion. Treated animals were divided into 4 groups, based on whether treatment started 2 days before surgery (n=3), upon reperfusion (n=3), 4 hours after stroke onset (n=4) or 6 hours after stroke onset (n=4). 3 control animals and 3 treated animals were killed at 24 h after stroke onset and processed for histology. In 3 control animals and 5 treated animals (1 from group III and 4 from group IV) a tail vein was cannulated at day 1 for injection of the NMR-contrast agent dimegluminegadopentate (Gd-DTPA).

NMR Experiments

A single-scan diffusion-trace MRI sequence (4 b values: 100-1780 S/mm², repetition time (TR)=2 s, echo time (TE)=100 ms, number of transients (NT)=2) was used to generate quantified images of tissue water trace apparent diffusion coefficient (ADC). T₂-weighted images were acquired using a multi-echo, sequence (8 TEs: 17.5 ms+7×17.5 ms, TR=2 s, NT=2). Before bolus injection a pre-contrast T₁-weighted dataset (TR=0.65 sec, TE=0.0125 sec, NT=2) was acquired and 15 min. after bolus injection, identical but contrast-enhanced T₁-weighted images were acquired. Both the, T₂-weighted and the diffusion-weighted datasets (3.2×3.2 cm² FOV, 128×64 matrix, zero-filled to 256×256) consisted of 8 consecutive, 1.7-mm thick slices, with 0-mm slice gap. The pre-, and post-contrast T₁-weighted images were acquired with a 128×128 matrix and zero filled to 512×512. To minimize interference at the slice boundaries, slices were, acquired in alternating order (1,3,5,7,2,4,6,8), thus maximizing the time between excitation of two neighbouring slices. For the diffusion-weighted imaging we used a double spin-echo pulse sequence with four pairs of bipolar gradients with specific predetermined signs in each of the three orthogonal directions as recently, published. The combination of gradient directions leads to cancellation of all off-diagonal tensor elements, effectively measuring the trace of the diffusion tensor. This provides unambiguous and rotationally invariant ADC values in one experiment, circumventing the need for three separate experiments. To minimize the inherently high sensitivity of diffusion-weighted imaging to motion, the acquisition was triggered to the respiration cycle. Diffusion weighted and T₂-weighted-datasets were acquired at 1, 7, and 21 days after stroke onset.

Immunohistochemistry

The following mouse-α-rat mAb were used. (αMHC class II, IgG1 isotype; Serotec, Oxford, UK), 1A29 (αICAM-1; IgG1 isotype; gift from Dr. T. Tamatani), 5F10 (αVCAM-1; IgG2a isotype; gift from Dr. R. Lobb, Biogen, Inc., Cambridge, Mass., USA) and HIS48 (αNeutrophil; IgGM isotype; Serotec, Oxford, UK). All stainings were performed at the dept. of Molecular Biology, VUMC, Amsterdam, NL.

Rats were sacrificed at 24 h hours after stroke onset. Brains were removed, snap-frozen and stored at −80° C. Cryostat sections (8 mm) were melted on gelatin coated glass slides and air-dried. Slides were fixed in acetone (10 min) and incubated with appropriate dilutions of mAb (2 μg/ml). As secondary antibody, a rabbit-a-mouse F(ab′)₂ conjugated to alkaline phosphatase (AF, Dako, Denmark) was used. Antibodies and conjugates were diluted in PBS with fetal calf serum (FCS). Omission of the primary antibody served as a negative control. After incubation-with the primary antibody (60 min), slides were rinsed in PBS, incubated with conjugate (60 min) and washed again in PBS. AF activity was demonstrated by incubation with AS-BI substrate in 0.1 M Tris pH 8.7 buffer for 10 minutes. After mounting the sections in mouting medium they were examined with a NIKON Eclipse E800 microscope and recordings were made with a NIKON DXM1200 camera.

Data Analysis

ADC and T₂ maps were generated by mono-exponential fitting using IDL (Research Systems, Boulder, Colo. USA). Parametric images were analysed in anatomic regions of interest using in-house software. Calculations of volumes of affected tissue were based on ipsilateral ADC or T₂, differing more than 20% (corresponding to >2×SD) from the mean value in the contralateral hemisphere.

Statistical analysis was done using a linear mixed effects model (in S-PLUS 2000 Professional Edition Release 3, Mathsoft Inc., USA), with Rat as random effect and Day and Treatment as fixed effects. After showing significance of all treatment effects, further statistical analysis was carried out using SPSS 10.0 (SPSS Inc., Chicago, USA). Reported p-values correspond to two-tailed Student's t-tests for the dichotomized comparisons. (Treatment-start-2-days-prior vs Treatment-start-after-onset) or one-way, ANOVA, where appropriate, with correction for multiple comparisons.

Results

General Conditions

After surgery, body weight of all animals showed an initial decrease in the first postoperative days, whereafter it increased.

In two treated animals a necrotic patch, about 7.5 mm in diameter, developed in the skin around the site of IFN-injection. The lesion was not painful to the touch and did not hinder the animals in feeding or other behavior. For the remaining days injection was continued at a different site, where, in these two animals, necrosis did not occur.

Magnetic Resonance Imaging

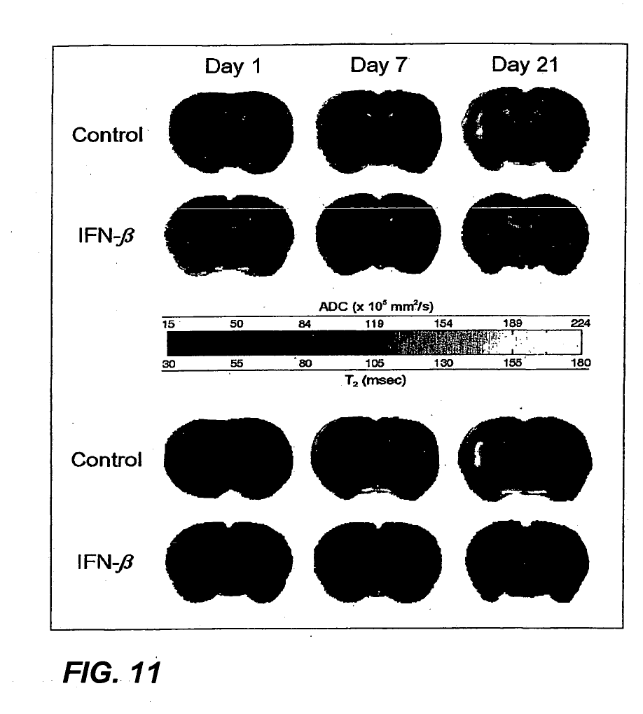

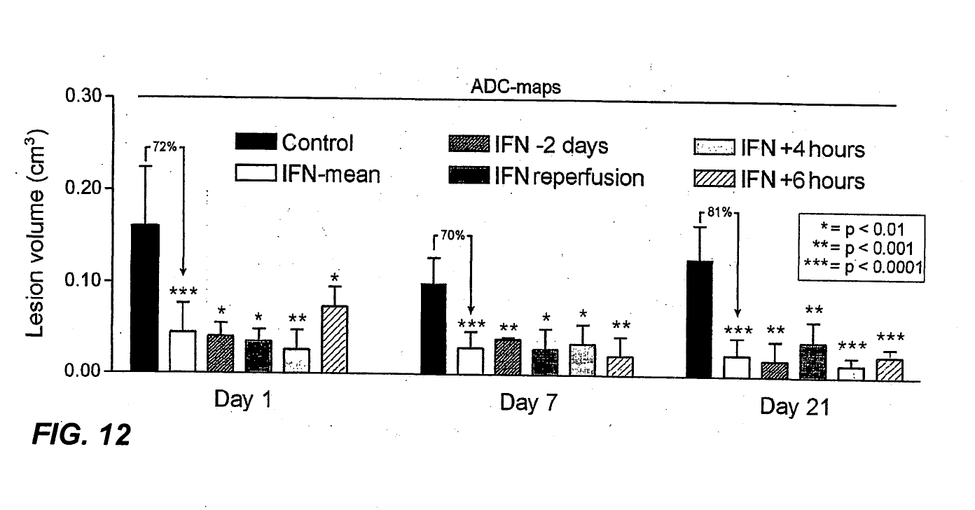

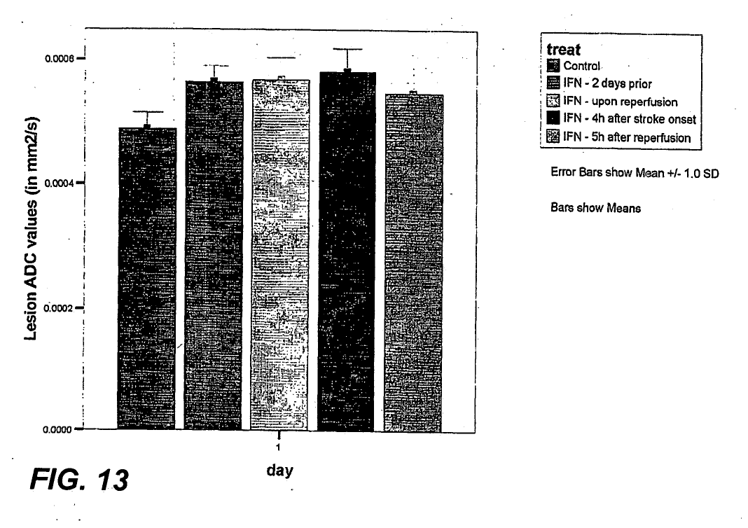

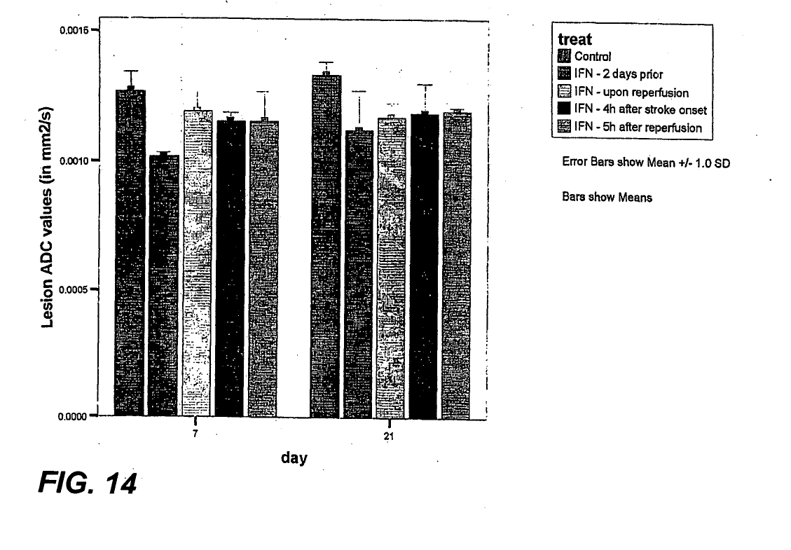

In control animals, both the cortex and a large part of the caudate putamen of the ipsilateral hemisphere were affected, whereas in IFN-treated animals cytotoxic cell swelling was restricted to a smaller area of the caudate putamen and to only a small rim of cortex, if at all, (FIG. 11). At this timepoint the volume of the lesion on ADC maps was ˜70% smaller (P<0.01) in IFN-treated animals than in controls (FIG. 12). Also, the severity of tissue damage, as determined from the extent of ADC reduction, was 14% less (P<0.001) in IFN-treated animals than in control animals (Table 1). There was no significant difference in ADC data between starting IFN-treatment up to 6 hours after stroke had occured or starting treatment 2 days before surgery.

Table 1. Mean lesion T₂ value of the control group and all treatment groups, on days 1, 7 and 21 as calculated from T₂ maps. All values were creased compared to normal tissue (0.055 s); control animals showed a stronger increase in lesion T₂ value.

Mean lesion ADC and T₂-values for all treatment groups on all days. The extent of ADC-decrease or T₂-increase is a measure for the severity of the lesion, irrespective of its size. The ischemia-induced decreases in ADC and increase in T₂ is attenuated by IFN treatment. *P<0.05 vs Ischemic Control, **P<0.01 vs Ischemic Control, ***P 0.001vs Ischemic-Control.

TABLE 1

| | Day 1 | | Day 7 | | Day 21 | |
|---|---|---|---|---|---|---|
| | ADC (×10⁻⁵ mm²/s) | T₂ (msec) | ADC (×10⁻⁵ mm²/s) | T₂ (msec) | ADC (×10⁻⁵ mm²/s) | T₂ (msec) |
| IFN-β | | | | | | |
| Healthy Control | 71.6 ± 4.0 | 53.4 ± 0.4 | 71.8 ± 2.5 | 54.5 ± 1.1 | 72.8 ± 1.4 | 53.6 ± 2.6 |
| Ischemic Control | 48.8 ± 2.6 | 81.4 ± 6.1 | 127.1 ± 7.4 | 85.6 ± 6.1 | 133.8 ± 4.9 | 93.4 ± 11.3 |
| 2 Days prior | 56.3 ± 2.6* | 68.5 ± 3.6** | 102.1 ± 1.2* | 73.3 ± 3.5* | 112.5 ± 1.5* | 72.4 ± 1.7* |
| Upon reperfusion | 56.8 ± 3.5** | 73.5 ± 7.5* | 119.2 ± 7.6 | 70.1 ± 1.0** | 117.2 ± 5.8* | 80.3 ± 6.3 |
| 4 Hours after onset | 58.1 ± 3.8** | 81.0 ± 3.2 | 115.9 ± 3.2* | 78.7 ± 2.1 | 119.1 ± 1.3* | 80.2 ± 12.6 |
| 6 Hours after onset | 54.6 ± 4.3* | 77.6 ± 2.6 | 115.8 ± 11.4* | 78.8 ± 9.3 | 119.9 ± 1.2* | 76.8 ± 10.6* |
| IFN-mean | 56.4 ± 3.5*** | 75.8 ± 6.1 | 116.7 ± 7.1** | 76.6 ± 6.5** | 117.6 ± 7.9** | 77.9 ± 8.5** |

* = p < 0.05
** = p < 0.01
*** = p < 0.001

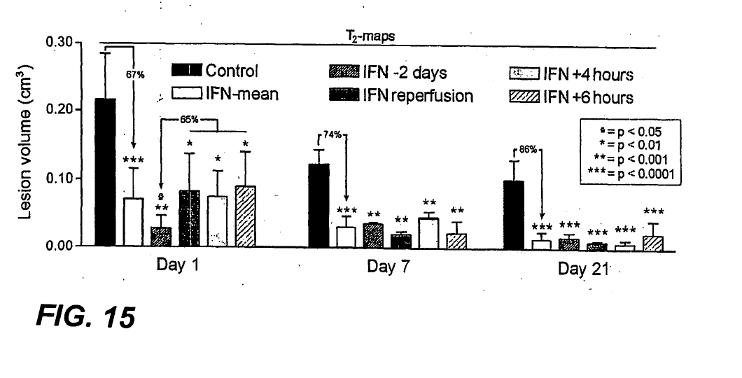

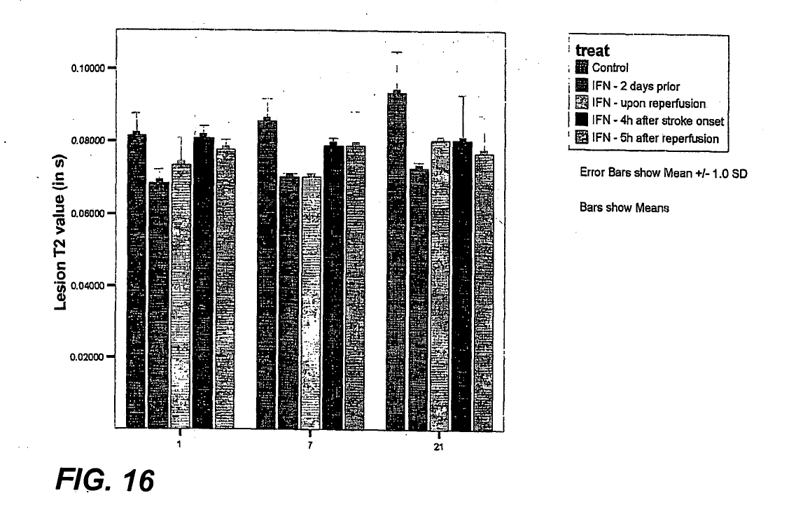

T₂ maps, calculated from T₂-weighted datasets, showed the presence of vasogenic edema in the affected tissue, the volume of which was ˜67% smaller (P<0.05) in IFN-treated animals compared to controls (FIGS. 11 and 15). This difference was also demonstrated by the midline-shift caused by the swelling of brain tissue, which was absent or much less pronounced in IFN-treated animals than in controls.

We subsequently analysed the effect of starting IFN treatment, 2 days before surgery or after stroke had occurred. In terms of the ADC data there was no difference between prophylactic treatment and post-hoc treatment. In terms of the T₂ data, prophylactically treated animals had ˜65% smaller lesion volumes on day 1 (P<0.05, FIG. 15) and the average lesion T₂-value was 12% tower (P<0.05; Table 1) than that of animals treated after stroke onset. From day 7 onwards, the effect of starting treatment up to 6 hours after stroke was similar to that of starting treatment 2 days before surgery.

On day 21, the last measurement day, ADC-values had increased to supranormal values, which are indicative of vasogenic edema and tissue loss (FIGS. 11 and 12). The infarct size on ADC maps was ˜80% smaller (P<0.0001) in IFN-treated animals than in controls. T₂ data acquired on day 21 confirmed the presence of vasogenic edema and tissue loss (FIG. 11). Infarct size as determined from T₂ maps was ˜85% smaller (P<0.0001) in IFN-treated animals than in controls (FIG. 15).

There was no significant difference between starting treatment 2 days prior to the insult, starting upon reperfusion or starting 4 or 6 hours after reperfusion, the latest timepoint tested, indicating a treatment window of 6 hours after onset of stroke and possibly longer.

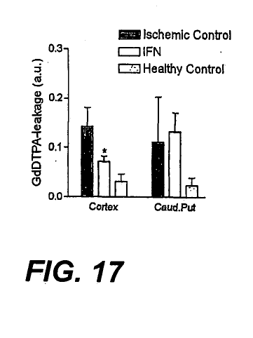

The BBB is the functional sum of the endothelial cells, pericytes, astrocytes and extra-cellular matrix molecules that surround the blood flowing through the brain. Opening of the BBB may result from H/I related inflammation. IFN has been reported to prevent opening of the BBB, both in the rat EAE model and in MS patients. To study the effect of IFN on BBB integrity, the leakage of the intravascular contrast agent Gd-DTPA was determined at 24 h after stroke onset by subtraction of pre- and post-contrast T₁-weighted images. The cortical BBB was better preserved in IFN-treated animals than in controls, as evidenced by a 49% decrease in Gd-DTPA-induced signal enhancement (P<0.05, FIG. 17).

Immunohistochemistry

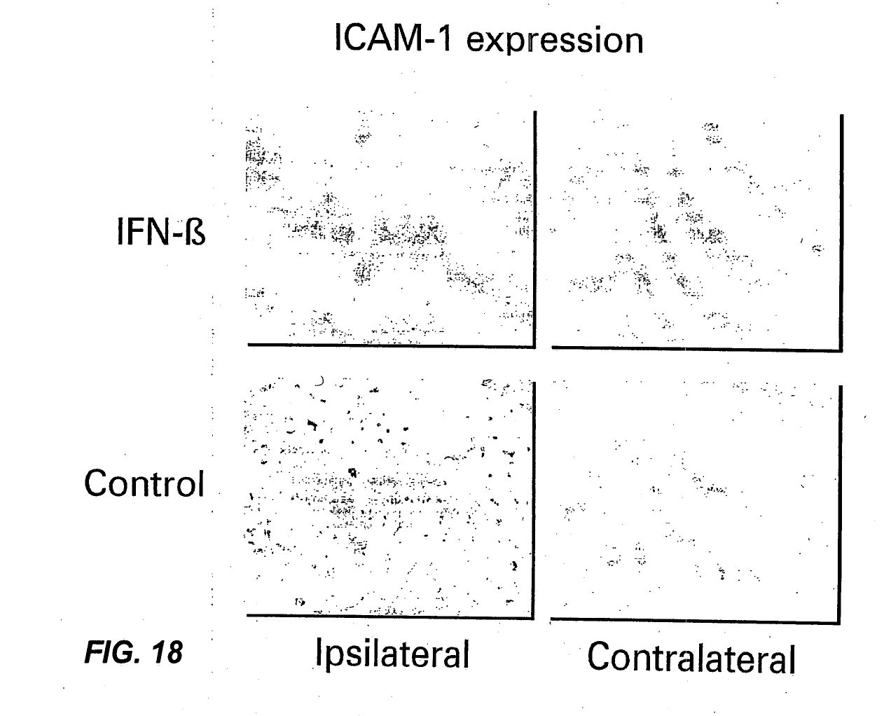

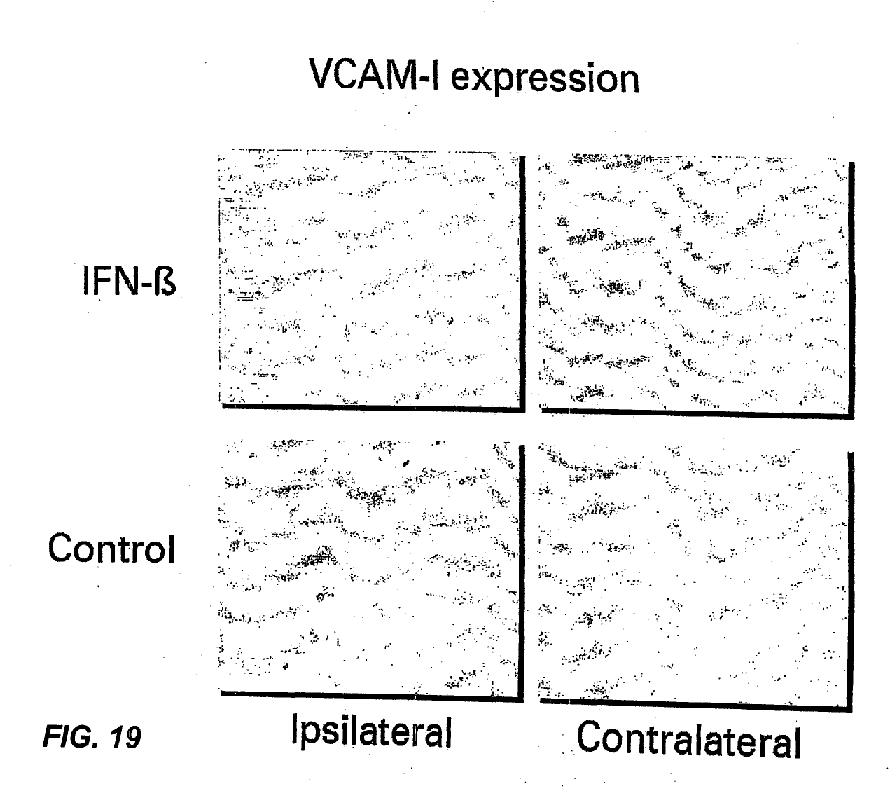

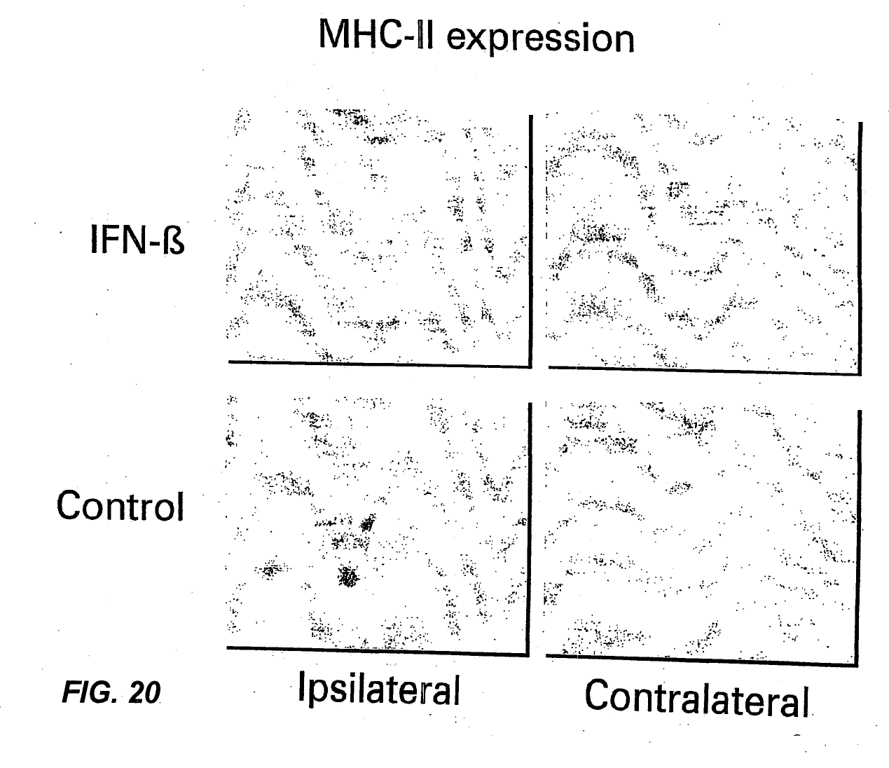

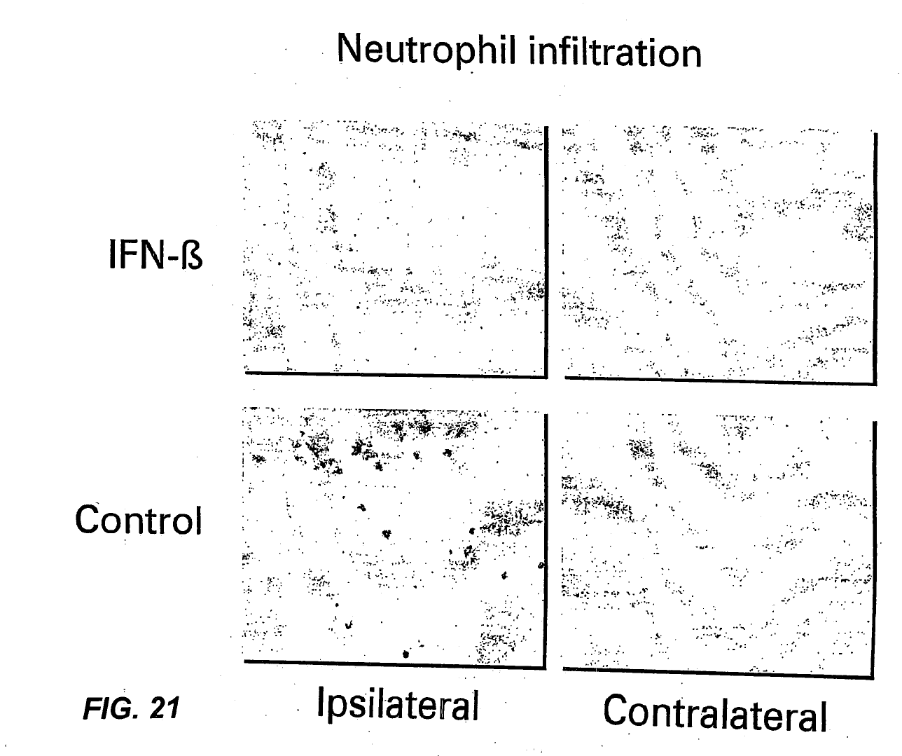

The adhesion molecules ICAM-1 and VCAM-1 were expressed at low levels in the contralateral hemisphere of both control and IFN treated animals. At 24 hours after stroke onset there was a strong upregulation of both ICAM-1 and VCAM-1 in the ipsilateral hemisphere of control animals. This upregulation was reduced in IFN treated animals (FIGS. 18 and 19). A similar pattern was seen for MHC-II expression. No MHCII was seen in the contralateral hemispheres of both control and IFN treated animals. In the ipsilateral hemisphere of control animals activated cells, expressing MHC-II, were present. This activation was prevented by IFN treatment (FIG. 20). Immunostaining for HIS48 to detect the presence of neutrophilic infiltrate showed the absence of infiltration in the contralateral hemispheres of all animals. The ipsilateral hemisphere of control animals showed clear neutrophil inflammatory infiltrate. IFN treatment reduced infiltration (FIG. 21).

### REFERENCES

Arnason B G (1999) Treatment of multiple sclerosis with interferon beta Biomed Pharmacother 53:344-350.

Barone F C, Feuerstein G Z (1999) Inflammatory mediators and stroke: new opportunities for novel therapeutics. J Cereb Blood Flow Metab 19:819-834.

Bona E, Andersson A L; Blomgren K, Gilland E, Puka-Sundvall M, Gustafson K, Hagberg H (1999) Chemokine and inflammatory cell response to hypoxia-ischemia in immature rats. Pediatr Res 45:500-509.

Bosca L, Bodelon O G, Hortelano S, Casellas A, Bosch F (2000) Anti-inflammatory action of type I interferons deduced from mice expressing interferon beta. Gene Ther 7:817-825.

Bowes M P, Rothlein R, Fagan S C, Zivin J A (1995) Monoclonal antibodies preventing leukocyte activation reduce experimental neurologic injury and enhance efficacy of thrombolytic therapy. Neurology 45:815-819.

Brint S, Jacewicz M, Kiessling M, Tanabe J, Pulsinelli W (1988) Focal brain ischemia in the rat: methods for reproducible neocortical infarction using tandem occlusion of the distal middle cerebral and ipsilateral common carotid arteries. J Cereb Blood Flow

Calabresi P A, Pelfrey C M, Tranquill L R, Maloni H. McFarland H F (1997) VLA-4 expression on peripheral blood lymphocytes is downregulated after treatment of multiple sclerosis with interferon beta Neurology 49:1111-1116.

Calabresi P A, Prat A, Biernacki K, Rollins J, Antel J P (2001) T lymphocytes conditioned with Interferon beta induce membrane and soluble, VCAM on human brain endothelial cells J Neuroimmunol 115:161-167.

Chabot S, Yong V W (2000). Interferon beta-1b increases interleukin-10 in a model of T cell-microglia interaction relevance to MS. Neurology 55:1497-1505.

Chabot S, Williams G, Yong V W (1997) Microglial production of TNF-alpha is induced by activated T lymphocytes. Involvement of VLA-4 and inhibition by interferonbeta-1b. J Clin Invest 100:604-612.

de Graaf R A, Braun K P, Nicolay K (2001) Single-shot diffusion trace (1) H NMR spectroscopy Magn Reson Med 45:741-748.

Defazio G, Livrea P, Giorelli M, Martino D, Roselli F, Ricchiuti F, Trojano M (2000) Interferon beta-1a downregulates TNFalpha-induced intercellular adhesion molecule 1 expression on brain microvascular endothelial cells through a tyrosine kinase-dependent pathway. Brain Res 881:227-230.

Deguchi M, Sakuta H, Uno K, Inaba K, Muramatsu S (1995) Exogenous and endogenous type I interferons inhibit interferon-gamma-induced nitric oxide production and nitric oxide synthase expression in murine peritoneal macrophages. J Interferon Cytokine Res 15:977-984.

Der S D, Zhou A, Williams B R, Silverman R H (1998) Identification of genes differentially regulated by interferon alpha, beta, or gamma using oligonucleotide arrays. Proc Natl. Acad Sci USA 95:15623-15628.

Dietrich W D, Busto R, Bethea J R, (1999) Postischemic hypothermia and IL-10 treatment provide long-lasting neuroprotection of CA1 hippocampus following transient global ischemia in rats. Exp Neurol 158:444-450.

Ehrlich L C, Hu S, Sheng W S, Sutton R L, Rockswold G L, Peterson P K, Chao C C (1998) Cytokine regulation of human microglial cell IL-8 production. J Immunol 160:1944-1948.

Fischer S, Liu M, MacLean A A, de Perrot M, Ho M, Cardella J A, Zhang XM, Bai XH, Suga M, Imai Y, Keshavjee S (2001) In vivo transtracheal adenovirus-mediated transfer of human interleukin-10 gene to donor lungs ameliorates ischemia-reperfusion injury and improves early posttransplant graft function in the rat. Hum Gene Ther. 12:1518-1526.

Gayo A, Mozo L, Suarez A, Tunon A, Lahoz C, Gutierrez C (1999) Interferon beta-1b treatment modulates TNFalpha and IFNgamma spontaneous gene expression in MS. Neurology 52:1764-1770.

Geng Y, Gulbins E, Altman A, Lotz M (1994) Monocyte deactivation by interleukin 10 via inhibition of tyrosine kinase activity and the Ras signaling pathway. Proc Natl Acad Sci U S A 91:8602-860.6.

Gross C E, Bednar M M, Howard D B, Sporn M B (1993) Transforming growth factor-beta 1 reduces infarct size after experimental cerebral ischemia in a rabbit model. Stroke 24:558-562. Guthikonda P, Baker J, Mattson D H (1998) Interferon-beta-1-b (IFN-B) decreases, induced nitric oxide (NO) production by a human astrocytoma cell line. J Neuroimmunol 82:133-139.

Halterman M W, Miller C C, Federoff H J (1999) Hypoxia-inducible factor-1.alpha mediates hypoxia-induced delayed neuronal death that involves p53. J Neurosci 19:6818-6824.

Howard M, O'Garra A (1992) Biological properties of interleukin 10. Immunol Today 13:198-200.

Hua L L, Liu J S, Brosnan C F, Lee S C (1998) Selective inhibition of human glial inducible nitric oxide synthase by interferon-beta: implications for multiple sclerosis. Ann Neurol 43:384-387.

Huynh H K, Oger J, Dorovini-Zis K (1995) Interferon-beta downregulates interferon-gamma-induced class II MHC molecule expression and morphological changes in primary cultures of human brain microvessel endothelial cells. J Neuroimmunol 60:63-73.

Iarlori C, Reale M, Lugaresi A, De Luca G, Bonanni L, Di Iorio A, Feliciani C, Conti P, Gambi D (2000) RANTES production and expression is reduced in relapsing-remitting multiple sclerosis patients treated with interferon-beta-1b. J Neuroimmunol 107:100-107.

Jungo F, Dayer J M, Modoux C, Hyka N, Burger D (2001) Ifn-beta inhibits the ability of t lymphocytes to induce tnf-alpha and il-1beta production in monocytes upon direct cell-cell contact. Cytokine 14:272-282.

Kallmann B A, Hummel V, Lindenlaub T, Ruprecht K, Toyka K V, Rieckmann P (2000) Cytokine-induced modulation of cellular adhesion to human cerebral endothelial cells is mediated by soluble vascular cell adhesion molecule-1. Brain 123:687-697.

Liu Z, Pelfrey C M, Cotleur A, Lee J C, Rudick R A (2001) Immunomodulatory effects of interferon beta-1a in multiple sclerosis. J Neuroimmunol 112:153-162.

Lopez-Collazo E, Hortelano S, Rojas A, Bosca L (1998) Triggering of peritoneal macrophages with IFN-alpha/beta attenuates the expression of inducible nitric oxide synthase through a decrease in NF-kappaB activation. J Immunol 160:2889-2895.

Masure S, Proost P, Van Damme J, Opdenakker G (1991) Purification and identification of 91-kDa neutrophil gelatinase. Release by the activating peptide interleukin-8. Eur J Biochem 198:391-398.

McNeill H, Williams C, Guan J, Dragunow M, Lawlor P, Sirimanne E, Nikolics K, Gluckman P (1994) Neuronal rescue with transforming growth factor-beta 1 after hypoxic ischaemic brain injury. Neuroreport 5:901-904.

Miller A, Lanir N, Shapiro S, Revel M, Honigman S, Kinarty A, Lahat N (1996) Immunoregulatory effects of interferon-beta and interacting cytokines on human vascular endothelial cells. Implications for multiple sclerosis autoimmune diseases J Neuroimmunol 64:151-161.

Muraro P A, Leist T, Bielekova B, McFarland HF (2000) VLA-4/CD49d downregulated on primed T lymphocytes during interferon-beta therapy in multiple sclerosis. J Neuroimmunol 111:186-194.

Nicoletti F, Di Marco R, Patti E, Reggio E, Nicoletti A, Zaccone P, Stivala F, Meroni P L, Reggio A (1998) Blood levels of transforming growth factor-beta 1 (TGF-beta1) are elevated in both relapsing remitting and chronic progressive multiple sclerosis (MS) patients and are further augmented by treatment with interferon-beta 1b (IFN-beta1b). Clin Exp Immunol 113:96-99.

Oliveira I C, Sciavolino P J, Lee T H, Vilcek J (1992) Downregulation of interleukin 8 gene expression in human fibroblasts: unique mechanism of transcriptional inhibition by interferon. Proc Natl Acad Sci USA 89:9049-9053.

Oliveira I C, Mukaida N, Matsushima K, Vilcek J (1994) Transcriptional inhibition of the interleukin-8 gene by interferon is mediated by the NF-kappa B site. Mol Cell. Biol 14:5300-5308.

Ossege L M, Sindern E, Patzold T, Malin J P (2001) Immunomodulatory effects of interferon-beta-1b in patients with multiple sclerosis. Int Immunopharmacol 1:1085-1100.

Ozenci V, Kouwenhoven M, Huang Y M, Xiao B, Kivisakk P, Fredrikson S, Link H (1999) Multiple sclerosis: levels of interleukin-10-secreting blood mononuclear cells are low in untreated patients but augmented during interferon-beta-1b treatment. Scand J Immunol 49:554-561.

Perini P, Tiberio M, Sivieri S, Facchinetti A, Biasi G, Gallo P (2000) Interleukin-1 receptor antagonist, soluble tumor necrosis factor-alpha receptor type I and II, and soluble E-selectin serum levels in multiple sclerosis patients receiving weekly intramuscular injections of interferon-beta1a Eur Cytokine Netw 11:81-86.

Plunkett J A, Yu C G, Easton J M, Bethea J R, Yezierski R P (2001) Effects of interleukin-10 (IL-b) on pain behavior and gene expression following excitotoxic spinal cord injury in the rat Exp Neurol 168:144-154.

Soilu-Hanninen M, Salmi A, Salonen R (1995) Interferon-beta downregulates expression of VLA-4 antigen and antagonizes interferon-gamma-induced expression of HLA-DQ on human peripheral blood monocytes. J Neuroimmunol 60:99-106.

Spera P A, Ellison J A, Feuerstein G Z, Barone F C (1998) IL-10 reduces rat brain injury following focal stroke. Neurosci Lett 251:189-192.

Stone L A, Frank J A, Albert P S, Bash C, Smith M E, Maloni H, McFarland H F (1995) The effect of interferon-beta on blood-brain barrier disruptions demonstrated by contrast enhanced magnetic resonance imaging in relapsing-remitting multiple sclerosis. Ann Neurol 37:611-619.

Stuve O, Chabot S, Jung S S, Williams G, Yong V W (1997) Chemokine-enhanced migration of human peripheral blood-mononuclear cells is antagonized by interferon beta-1b through an effect on matrix metalloproteinase-9. J Neuroimmunol 80:38-46.

Sumii T, Lo E H (2002) Involvement of matrix metalloproteinase in thrombolysis-associated hemorrhagic transformation after embolic focal ischemia in rats. Stroke 33:83,1-836.

Szczepanik A M, Funes S, Petko W, Ringheim GE (2001) IL-4, IL-10 and IL-13 modulate A beta(1-42)-induced cytokine and chemokine production in primary murine microglia and a human monocyte cell line. J Neuroimmunol 113:49-62.

Trojano M, Defazio G, Avolio C, Paolicelli D, Giuliani F. Giorelli M, Livrea P (2000) Effects of rIFN-beta-1b on serum circulating ICAM-1 in relapsing remitting multiple sclerosis and on the membrane bound ICAM-1 expression on brain microvascular endothelial cells. J Neurovirol 6 Suppl 2S47-51.

Tsuruma T, Yagihashi A, Tarumi K, Hirata K (1998) Anti-rat IL-8 (CINC) monoclonal antibody administration reduces ischemia-reperfusion injury in small intestine. Transplant Proc. 30:2644-2645.

Tuohy V K, Yu M, Yin L, Mathisen P M, Johnson J M, Kawczak J A (2000) Modulation of the IL-10/IL-12 cytokine circuit by interferon-beta inhibits the development of epitope spreading and disease progression in murine autoimmune encephalomyelitis. J Neuroimmunol 111:55-63.

van der Meide P H, de Labie M C, Ruuls S R, Groenestein R J, Botman C A, Olsson T, Dijkstra C D (1998) Discontinuation of treatment with IFN-beta leads to exacerbation of experimental autoimmune encephalomyelitis in Lewis rats. Rapid reversal of the antiproliferative activity of IFN-beta and excessive expansion of autoreactive T cells as disease promoting mechanisms. J Neuroimmunol 84:14-23.

von Hundelshausen P, Weber K S, Huo Y, Proudfodt A E, Nelson P J, Ley K, Weber C (2001) RANTES deposition by platelets triggers monocyte arrest on inflamed and atherosclerotic endothelium. Circulation 103:1772-1777.

Waubant E, Gee L, Miller K, Stabler G, Goodkin D (2001a) Ifn-beta1a may increase serum levels of timp-1 in patients with relapsing-remitting multiple sclerosis. J Interferon Cytokine Res 21:181-185.

Waubant E, Gee L, Bacchetti P, Sloan R, Cotleur A, Rudick R, Goodkin D (2001b) Relationship between serum levels of IL-10, MRI activity and interferon beta-1a therapy in patients with relapsing remitting MS. J Neuroimmunol 112-139-145.

Zang Y C, Halder J B, Samanta A K, Hong J, Rivera VI.VI, Zhang J Z (2001) Regulation of chemokine receptor CCR5 and production of RANTES and MIP-1alpha by interferon-beta. J Neuroimmunol 112:174-180.

Zhang R L, Zhang Z G, Chopp M (1999a) Increased therapeutic efficacy with rt-PA and anti-CD18 antibody treatment of stroke in the rat. Neurology 52:273-279.

Zhang R L, Zhang Z G, Chopp M, Zivin J A (1999b) Thrombolysis with tissue plasminogen activator alters adhesion molecule expression in the ischemic rat brain. Stroke 30:624-629.

Zou L P, Ma D H, Wei L, van der Meide P H, Mix E, Zhu J (1999) IFN-beta suppresses experimental autoimmune neuritis in Lewis rats by inhibiting the migration of inflammatory cells into peripheral nervous tissue. J Neurosci Res 56:123-130.

What is claimed is:

1. A method for reducing cellular damage in tissue that has suffered from or is suffering from hypoxia and/or ischemia and/or inflammation in an individual said method comprising administering to the individual a dose of IFN type-I or a functional part, derivative and/or analogue thereof.

2. The method according to claim 1, wherein the H/I related inflammation results from a shortage in blood supply.

3. The method according to claim 1, wherein the H/I related inflammation is restricted to a body part of the individual.

4. The method according to claim 2, wherein the H/I related inflammation is restricted to a body part of the individual.

5. The method according to claim 3 or claim 4, wherein the body part comprises the brain, the spinal cord, the heart, a transplanted organ, and/or a limb.

6. The method according to claim 5, wherein the H/I related inflammation is restricted to a part of the brain, heart or brain and heart.

7. The method according to claim 6, wherein the H/I related inflammation is restricted to a part of the brain.

8. The method according to claim 1, claim 2, claim 3, claim 4, claim 5, claim 6, or claim 7, wherein the H/I related inflammation is induced by an obstruction of a blood vessel.

9. A method for treating an H/I related inflammation in an individual having at least one blood vessel obstruction causing ischemia in tissue that is situated downstream from said obstruction, said method comprising:
administering to the individual a dose of IFN type-I or a functional part, derivative and/or analogue thereof.

10. A method for at least in part improving blood flow in post-ischaemic tissue, said method comprising administering, to an individual having said tissue, IFN type-I or a functional part, derivative, and/or analogue thereof.

11. A method for at least in part preventing cell death in post-ischaemic tissue, said method comprising administering, to an individual having said tissue, IFN type-I or a functional part, derivative and/or analogue thereof.

12. A method of treating impairment of blood flow recovery in a subject, said method comprising: administering to the subject an IFN type-I or a functional part, derivative and/or analogue thereof in a therapeutically effective amount in a pharmaceutically acceptable manner.

13. The method according to claim 12, wherein said impairment is in a capillary vessel.

14. The method according to claim 12 or claim 13, wherein said impairment is in the brain.

15. A method for treating a hypoxia/ischemia (H/I) related inflammation in an individual, said method comprising:
administering to the individual, IFN type-I or a functional part, derivative and/or analogue thereof.

16. The method according to claim 15, wherein the H/I related inflammation results from a shortage in blood supply.

17. The method according to claim 15, or claim 16, wherein the H/I related inflammation is restricted to a body part of the individual.

18. The method according to claim 17, wherein the body part comprises the brain, the spinal cord, the heart, a transplanted organ, and/or a limb.

19. The method according to claim 18, wherein the H/I related inflammation is restricted to a part of the brain, heart, or brain and heart.

20. The method according to claim 19, wherein the H/I related inflammation is restricted to a part of the brain.

21. The method according to claim 15, claim 16, claim 17, claim 18, claim 19, and claim 20, wherein the H/I related inflammation is induced by an obstruction of a blood vessel.
